(19) United States
(12) Patent Application Publication
(10) Pub. No.: US 2005/0032068 A1
(43) Pub. Date: Feb. 10, 2005

(54) Sugar and backbone-surrogate-containing oligomeric compounds and compositions for use in gene modulation

(76) Inventors: Thazha P. Prakash, Carlsband, CA (US); Muthiah Manoharan, Weston, MA (US)

Correspondence Address: WOODCOCK WASHBURN LLP, ONE LIBERTY PLACE - 46TH FLOOR, PHILADELPHIA, PA, 19103, US

(21) Appl. No.: 10/634,483

(22) Filed: Aug. 5, 2003

(51) Int. Cl. C12Q 1/68; C07H 21/02; A61K 48/00

(52) U.S. Cl. 435/6; 514/44; 536/23.1

(57) ABSTRACT

Compositions comprising first and second oligomers are provided wherein at least a portion of the first oligomer is capable of hybridizing with at least a portion of the second oligomer, at least a portion of the first oligomer is complementary to and capable of hybridizing to a selected target nucleic acid, and at least one of the first or second oligomers includes a modified sugar and/or backbone modification. In some embodiments the modification is a peptide nucleic acid, a peptide nucleic acid mimic, a morpholino nucleic acid, hexose sugar with amide linkage, cyclohexenyl nucleic acid (CeNA), or an acyclic backbone moiety. Oligomer/protein compositions are also provided comprising an oligomer complementary to and capable of hybridizing to a selected target nucleic acid and at least one protein comprising at least a portion of an RNA-induced silencing complex (RISC), wherein at least one nucleotide of the oligomer has a modified sugar and/or backbone modification.

### FIELD OF THE INVENTION

The present invention provides modified oligomers that modulate gene expression via a RNA interference pathway. The oligomers of the invention include one or more modifications thereon resulting in differences in various physical properties and attributes compared to wild type nucleic acids. The modified oligomers are used alone or in compositions to modulate the targeted nucleic acids. In preferred embodiments of the invention, the modifications include peptide nucleic acids, peptide nucleic acid mimics, morpholino nucleic acids, oliogmers with hexose sugars and amide linkages, cyclohexenyl nucleic acids (CeNA) and acyclic backbone moieties.

### BACKGROUND OF THE INVENTION

In many species, introduction of double-stranded RNA (dsRNA) induces potent and specific gene silencing. This phenomenon occurs in both plants and animals and has roles in viral defense and transposon silencing mechanisms. This phenomenon was originally described more than a decade ago by researchers working with the petunia flower. While trying to deepen the purple color of these flowers, Jorgensen et al. introduced a pigment-producing gene under the control of a powerful promoter. Instead of the expected deep purple color, many of the flowers appeared variegated or even white. Jorgensen named the observed phenomenon “cosuppression”, since the expression of both the introduced gene and the homologous endogenous gene was suppressed (Napoli et al., Plant Cell, 1990, 2, 279-289; Jorgensen et al., Plant Mol. Biol., 1996, 31, 957-973).

Cosuppression has since been found to occur in many species of plants, fungi, and has been particularly well characterized in Neurospora crassa, where it is known as “quelling” (Cogoni and Macino, Genes Dev. 2000, 10, 638-643; Guru, Nature, 2000, 404, 804-808).

The first evidence that dsRNA could lead to gene silencing in animals came from work in the nematode, Caenorhabditis elegans. In 1995, researchers Guo and Kemphues were attempting to use antisense RNA to shut down expression of the par-1 gene in order to assess its function. As expected, injection of the antisense RNA disrupted expression of par-1, but quizzically, injection of the sense-strand control also disrupted expression (Guo and Kempheus, Cell, 1995, 81, 611-620). This result was a puzzle until Fire et al. injected dsRNA (a mixture of both sense and antisense strands) into C. elegans. This injection resulted in much more efficient silencing than injection of either the sense or the antisense strands alone. Injection of just a few molecules of dsRNA per cell was sufficient to completely silence the homologous gene's expression. Furthermore, injection of dsRNA into the gut of the worm caused gene silencing not only throughout the worm, but also in first generation offspring (Fire et al., Nature, 1998, 391, 806-811).

The potency of this phenomenon led Timmons and Fire to explore the limits of the dsRNA effects by feeding nematodes bacteria that had been engineered to express dsRNA homologous to the C. elegans unc-22 gene. Surprisingly, these worms developed an unc-22 null-like phenotype (Timmons and Fire, Nature 1998, 395, 854; Timmons et al., Gene, 2001, 263, 103-112). Further work showed that soaking worms in dsRNA was also able to induce silencing (Tabara et al., Science, 1998, 282, 430-431). PCT publication WO 01/48183 discloses methods of inhibiting expression of a target gene in a nematode worm involving feeding to the worm a food organism which is capable of producing a double-stranded RNA structure having a nucleotide sequence substantially identical to a portion of the target gene following ingestion of the food organism by the nematode, or by introducing a DNA capable of producing the double-stranded RNA structure (Bogaert et al., 2001).

The posttranscriptional gene silencing defined in Caenorhabditis elegans resulting from exposure to double-stranded RNA (dsRNA) has since been designated as RNA interference (RNAi). This term has come to generalize all forms of gene silencing involving dsRNA leading to the sequence-specific reduction of endogenous targeted mRNA levels; unlike co-suppression, in which transgenic DNA leads to silencing of both the transgene and the endogenous gene.

Introduction of exogenous double-stranded RNA (dsRNA) into Caenorhabditis elegans has been shown to specifically and potently disrupt the activity of genes containing homologous sequences. Montgomery et al. suggests that the primary interference affects of dsRNA are post-transcriptional. This conclusion being derived from examination of the primary DNA sequence after dsRNA-mediated interference and a finding of no evidence of alterations, followed by studies involving alteration of an upstream operon having no effect on the activity of its downstream gene. These results argue against an effect on initiation or elongation of transcription. Finally using in situ hybridization they observed that dsRNA-mediated interference produced a substantial, although not complete, reduction in accumulation of nascent transcripts in the nucleus, while cytoplasmic accumulation of transcripts was virtually eliminated. These results indicate that the endogenous mRNA is the primary target for interference and suggest a mechanism that degrades the targeted mRNA before translation can occur. It was also found that this mechanism is not dependent on the SMG system, an mRNA surveillance system in C. elegans responsible for targeting and destroying aberrant messages. The authors further suggest a model of how dsRNA might function as a catalytic mechanism to target homologous mRNAs for degradation. (Montgomery et al., Proc. Natl. Acad. Sci. USA, 1998, 95, 15502-15507).

Recently, the development of a cell-free system from syncytial blastoderm Drosophila embryos, which recapitulates many of the features of RNAi, has been reported. The interference observed in this reaction is sequence specific, is promoted by dsRNA but not single-stranded RNA, functions by specific mRNA degradation, and requires a minimum length of dsRNA. Furthermore, preincubation of dsRNA potentiates its activity demonstrating that RNAi can be mediated by sequence-specific processes in soluble reactions (Tuschl et al., Genes Dev., 1999, 13, 3191-3197).

In subsequent experiments, Tuschl et al, using the Drosophila in vitro system, demonstrated that 21- and 22-nt RNA fragments are the sequence-specific mediators of RNAi. These fragments, which they termed short interfering RNAs (siRNAs), were shown to be generated by an RNase III-like processing reaction from long dsRNA. They also showed that chemically synthesized siRNA duplexes with overhanging 3′ ends mediate efficient target RNA cleavage in the Drosophila lysate, and that the cleavage site is located near the center of the region spanned by the guiding siRNA. In addition, they suggest that the direction of dsRNA processing determines whether sense or antisense target RNA can be cleaved by the siRNA-protein complex (Elbashir et al., Genes Dev., 2001, 15, 188-200). Further characterization of the suppression of expression of endogenous and heterologous genes caused by the 21-23 nucleotide siRNAs have been investigated in several mammalian cell lines, including human embryonic kidney (293) and HeLa cells (Elbashir et al., Nature, 2001, 411, 494-498).

The Drosophila embryo extract system has been exploited, using green fluorescent protein and luciferase tagged siRNAs, to demonstrate that siRNAs can serve as primers to transform the target mRNA into dsRNA. The nascent dsRNA is degraded to eliminate the incorporated target mRNA while generating new siRNAs in a cycle of dsRNA synthesis and degradation. Evidence is also presented that mRNA-dependent siRNA incorporation to form dsRNA is carried out by an RNA-dependent RNA polymerase activity (RdRP) (Lipardi et al., Cell, 2001, 107, 297-307).

The involvement of an RNA-directed RNA polymerase and siRNA primers as reported by Lipardi et al. (Lipardi et al., Cell, 2001, 107, 297-307) is one of the many intriguing features of gene silencing by RNA interference. This suggests an apparent catalytic nature to the phenomenon. New biochemical and genetic evidence reported by Nishikura et al. also shows that an RNA-directed RNA polymerase chain reaction, primed by siRNA, amplifies the interference caused by a small amount of “trigger” dsRNA (Nishikura, Cell, 2001, 107, 415-418).

Investigating the role of “trigger” RNA amplification during RNA interference (RNAi) in Caenorhabditis elegans, Sijen et al revealed a substantial fraction of siRNAs that cannot derive directly from input dsRNA. Instead, a population of siRNAs (termed secondary siRNAs) appeared to derive from the action of the previously reported cellular RNA-directed RNA polymerase (RdRP) on mRNAs that are being targeted by the RNAi mechanism. The distribution of secondary siRNAs exhibited a distinct polarity (5′-3′; on the antisense strand), suggesting a cyclic amplification process in which RdRP is primed by existing siRNAs. This amplification mechanism substantially augmented the potency of RNAi-based surveillance, while ensuring that the RNAi machinery will focus on expressed mRNAs (Sijen et al., Cell, 2001, 107, 465-476).

Most recently, Tijsterman et al. have shown that, in fact, single-stranded RNA oligomers of antisense polarity can be potent inducers of gene silencing. As is the case for co-suppression, they showed that antisense RNAs act independently of the RNAi genes rde-1 and rde-4 but require the mutator/RNAi gene mut-7 and a putative DEAD box RNA helicase, mut-14. According to the authors, their data favor the hypothesis that gene silencing is accomplished by RNA primer extension using the mRNA as template, leading to dsRNA that is subsequently degraded suggesting that single-stranded RNA oligomers are ultimately responsible for the RNAi phenomenon (Tijsterman et al., Science, 2002, 295, 694-697).

Several recent publications have described the structural requirements for the dsRNA trigger required for RNAi activity. Recent reports have indicated that ideal dsRNA sequences are 21nt in length containing 2 nt 3′-end overhangs (Elbashir et al, EMBO (2001), 20, 6877-6887, Sabine Brantl, Biochimica et Biophysica Acta, 2002, 1575, 15-25.) In this system, substitution of the 4 nucleosides from the 3′-end with 2′-deoxynucleosides has been demonstrated to not affect activity. On the other hand, substitution with 2′-deoxynucleosides or 2′-OMe-nucleosides throughout the sequence (sense or antisense) was shown to be deleterious to RNAi activity.

Investigation of the structural requirements for RNA silencing in C. elegans has demonstrated modification of the internucleotide linkage (phosphorothioate) to not interfere with activity (Parrish et al., Molecular Cell, 2000, 6, 1077-1087.) It was also shown by Parrish et al., that chemical modification like 2′-amino or 5-iodouridine are well tolerated in the sense strand but not the antisense strand of the dsRNA suggesting differing roles for the 2 strands in RNAi. Base modification such as guanine to inosine (where one hydrogen bond is lost) has been demonstrated to decrease RNAi activity independently of the position of the modification (sense or antisense). Some “position independent” loss of activity has been observed following the introduction of mismatches in the dsRNA trigger. Some types of modifications, for example introduction of sterically demanding bases such as 5-iodoU, have been shown to be deleterious to RNAi activity when positioned in the antisense strand, whereas modifications positioned in the sense strand were shown to be less detrimental to RNAi activity. As was the case for the 21 nt dsRNA sequences, RNA-DNA heteroduplexes did not serve as triggers for RNAi. However, dsRNA containing 2′-F-2′-deoxynucleosides appeared to be efficient in triggering RNAi response independent of the position (sense or antisense) of the 2′-F-2′-deoxynucleosides.

In one study the reduction of gene expression was studied using electroporated dsRNA and a 25mer morpholino oligomer in post implantation mouse embryos (Mellitzer et al., Mehanisms of Development, 2002, 118, 57-63). The morpholino oligomer did show activity but was not as effective as the dsRNA.

A number of PCT applications have recently been published that relate to the RNAi phenomenon. These include: PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164.

U.S. Pat. Nos. 5,898,031 and 6,107,094, each of which is commonly owned with this application and each of which is herein incorporated by reference, describe certain oligonucleotide having RNA like properties. When hybridized with RNA, these oligonucleotides serve as substrates for a dsRNase enzyme with resultant cleavage of the RNA by the enzyme.

In another recently published paper (Martinez et al., Cell, 2002, 110, 563-574) it was shown that single stranded as well as double stranded siRNA resides in the RNA-induced silencing complex (RISC) together with elF2C1 and elf2C2 (human GERp950) Argonaute proteins. The activity of 5′-phosphorylated single stranded siRNA was comparable to the double stranded siRNA in the system studied. In a related study, the inclusion of a 5′-phosphate moiety was shown to enhance activity of siRNA's in vivo in Drosophilia embryos (Boutla, et al., Curr. Biol., 2001, 11, 1776-1780). In another study, it was reported that the 5′-phosphate was required for siRNA function in human HeLa cells (Schwarz et al., Molecular Cell, 2002, 10, 537-548).

In yet another recently published paper (Chiu et al., Molecular Cell, 2002, 10, 549-561) it was shown that the 5′-hydroxyl group of the siRNA is essential as it is phosphorylated for activity while the 3′-hydroxyl group is not essential and tolerates substitute groups such as biotin. It was further shown that bulge structures in one or both of the sense or antisense strands either abolished or severely lowered the activity relative to the unmodified siRNA duplex. Also shown was severe lowering of activity when psoralen was used to cross link an siRNA duplex.

Like the RNAse H pathway, the RNA interference pathway for modulation of gene expression is an effective means for modulating the levels of specific gene products and, thus, would be useful in a number of therapeutic, diagnostic, and research applications involving gene silencing. The present invention therefore provides oligomeric compounds useful for modulating gene expression pathways, including those relying on mechanisms of action such as RNA interference and dsRNA enzymes, as well as antisense and non-antisense mechanisms. One having skill in the art, once armed with this disclosure will be able, without undue experimentation, to identify preferred oligonucleotide compounds for these uses.

### SUMMARY OF THE INVENTION

In certain aspects, the invention relates to compositions comprising a first oligomer and a second oligomer, each having linked nucleosidic bases. At least a portion of the first oligomer is capable of hybridizing with at least a portion of the second oligomer, at least a portion of the first oligomer is complementary to and capable of hybridizing to a selected target nucleic acid, and at least one of the oligomers includes a a peptide nucleic acid, a peptide nucleic acid mimic, a morpholino nucleic acid, hexose sugar with amide linkage, cyclohexenyl nucleic acid (CeNA) or an acyclic backbone moiety.

In certain other embodiments, the invention is directed to oligonucleomer/protein compositions comprising an oligomer complementary to and capable of hybridizing to a selected target nucleic acid, and at least one protein comprising at least a portion of a RNA-induced silencing complex (RISC). The oligomer includes at least one nucleotide having a peptide nucleic acid, a morpholino nucleic acid, or an acyclic backbone moiety.

In other aspects, the invention relates to oligomershaving at least a first region and a second region where the first region is complementary to and capable of hybridizing with the second region, and at least a portion of the oligomer is complementary to and is capable of hybridizing to a selected target nucleic acid. The oligomer further includes at least one peptide nucleic acid, morpholino nucleic acids or acyclic backbone moiety.

The first and second oligomers preferably each have 10 to 40 nucleosidic bases. In other embodiments, each of the first and second oligomers have 18 to 30 nucleosidic bases. In yet other embodiments, the first and second oligomers have 21 to 24 nucleosidic bases.

Also provided by the present invention are pharmaceutical compositions comprising any of the above compositions or oligomeric compounds and a pharmaceutically acceptable carrier.

Methods for modulating the expression of a target nucleic acid in a cell are also provided, wherein the methods comprise contacting the cell with any of the above compositions or oligomeric compounds.

Methods of treating or preventing a disease or condition associated with a target nucleic acid are also provided, wherein the methods comprise administering to a patient having or predisposed to the disease or condition a therapeutically effective amount of any of the above compositions or oligomeric compounds.

### DETAILED DESCRIPTION OF THE INVENTION

The present invention provides oligomeric compounds useful in the modulation of gene expression. Although not intending to be bound by theory, oligomeric compounds of the invention modulate gene expression by hybridizing to a nucleic acid target resulting in loss of normal function of the target nucleic acid. As used herein, the term “target nucleic acid” or “nucleic acid target” is used for convenience to encompass any nucleic acid capable of being targeted including without limitation DNA, RNA (including pre-mRNA and mRNA or portions thereof) transcribed from such DNA, and also cDNA derived from such RNA. In a preferred embodiment of this invention modulation of gene expression is effected via modulation of a RNA associated with the particular gene RNA.

The invention provides for modulation of a target nucleic acid that is a messenger RNA. The messenger RNA is degraded by the RNA interference mechanism as well as other mechanisms in which double stranded RNA/RNA structures are recognized and degraded, cleaved or otherwise rendered inoperable.

The functions of RNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

Compounds of the Invention

The compounds of the invention comprise nucleosidic bases that are linked in a way that permits them to hybridize with an appropriate nucleic acid target. Any of the many linkages known in the art can be used, so long as at least one peptide nucleic acid, peptide nucleic acid mimic, morpholino nucleic acid, hexose sugar with amide linkage, cyclohexenyl nucleic acid (CeNA) or acyclic backbone moiety is employed. Thus, nucleosidic bases can be linked using pentofuranosyl sugars and phosphate internucleosid linkages (as in naturally-occurring nucleic acids) or pentofuranosyl sugars and one or more of the many known non-phosphate internucleoside linkages.

In PNA compounds, an amide containing backbone, in particular an aminoethylglycine backbone, is used in place of a sugar and internucleoside linkage. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; 5,719,262; and 6,395,474, each of which is herein incorporated by reference. Further teaching of PNA compounds can be found in Nielsen et al., Science, 1991, 254, 1497-1500.

Some compositions of the invention comprise a polyamide backbone bearing a plurality of ligands that are individually bound to aza nitrogen atoms located within said backbone, at least one of the ligands is a naturally occurring nucleobase, a non-naturally occurring nucleobase, a DNA intercalator, or a nucleobase-binding group. In some embodiments, the aza nitrogen atoms are separated from one another in said backbone by from 4 to 6 intervening atoms.

In certain embodiments, the PNA structures comprise at least one strand of the formula: 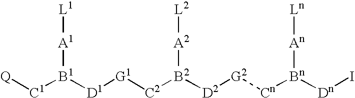
wherein:
n is at least 2,
each of L¹-Lⁿ is independently selected from the group consisting of hydrogen, hydroxy, (C₁-C₄)alkanoyl, naturally occurring nucleobases, non-naturally occurring nucleobases, aromatic moieties, DNA intercalators, nucleobase-binding groups, heterocyclic moieties, and reporter ligands, at least one of L¹-Lⁿ being a naturally occurring nucleobase, a non-naturally occurring nucleobase, a DNA intercalator, or a nucleobase-binding group;
each of A¹-Aⁿ is a single bond, a methylene group or a group of formula: 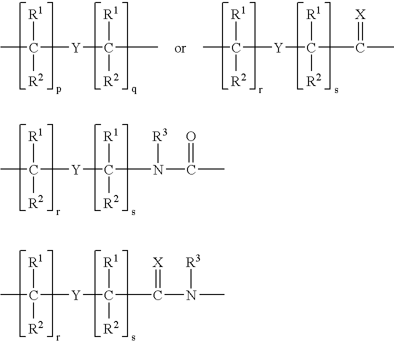
   where:
X is O, S, Se, NR³, CH₂ or C(CH₃)₂;
Y is a single bond, O, S or NR⁴;
each of p and q is zero or an integer from 1 to 5, the sum p+q being not more than 10;
each of r and s is zero or an integer from 1 to 5, the sum r+s being not more than 10;
each R¹ and R² is independently selected from the group consisting of hydrogen, (C₁-C₄)alkyl which may be hydroxy- or alkoxy- or alkylthio-substituted, hydroxy, alkoxy, alkylthio, amino and halogen; and
each R³ and R⁴ is independently selected from the group consisting of hydrogen, (C₁-C₄)alkyl, hydroxy- or alkoxy- or alkylthio-substituted (C₁-C₄)alkyl, hydroxy, alkoxy, alkylthio and amino;
each of B¹-Bⁿ is N or R³N⁺, where R³is as defined above;
each of C¹-Cⁿ is CR⁶R⁷, CHR⁶CHR⁷ or CR⁶R⁷CH₂, where R⁶ is hydrogen and R⁷ is selected from the group consisting of the side chains of naturally occurring alpha amino acids, or R⁶ and R⁷ are independently selected from the group consisting of hydrogen, (C₂-C₆)alkyl, aryl, aralkyl, heteroaryl, hydroxy, (C₁-C₆)alkoxy, (C₁-C₆)alkylthio, NR³R⁴ and SR⁵, where R³ and R⁴ are as defined above, and R⁵is hydrogen, (C₁-C₆)alkyl, hydroxy-, alkoxy-, or alkylthio-substituted (C₁-C₆)alkyl, or R⁶ and R⁷ taken together complete an alicyclic or heterocyclic system;
each of D¹-Dⁿ is CR⁶R⁷, CH₂CR⁶R⁷ or CHR⁶CHR⁷, where R⁶ and R⁷ are as defined above;
each of G¹-Gⁿ⁻¹ is —NR³CO—, —NR³CS—, —NR³SO— or —NR³SO²⁻, in either orientation, where R³ is as defined above;
Q is —CO₂H, —CONR′R″, —SO₃H or —SO₂NR′R″ or an activated derivative of —CO₂H or —SO₃H; and
I is —NHR′″R″″ or —NR′″C(O)R″″, where R′, R″, R′″ and R″″ are independently selected from the group consisting of hydrogen, alkyl, amino protecting groups, reporter ligands, intercalators, chelators, peptides, proteins, carbohydrates, lipids, steroids, oligonucleotides and soluble and non-soluble polymers.

In other embodiments, the PNA structures are of the formula: 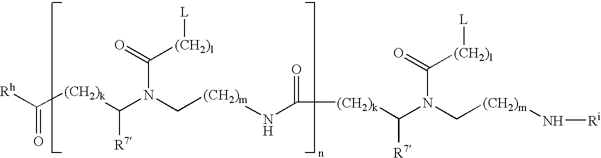
wherein:
each L is independently selected from the group consisting of hydrogen, phenyl, heterocyclic moieties, naturally occurring nucleobases, and non-naturally occurring nucleobases;
each R⁷′ is independently selected from the group consisting of hydrogen and the side chains of naturally occurring alpha amino acids;
n is an integer from 1 to 60,
each k and m is, independently, zero or 1;
each l is zero or an integer from 1 to 5;
R^{h} is OH, NH₂ or —NHLysNH₂; and
Rⁱ is H or COCH₃.

In yet other embodiments, the PNA structures are represented by the formula: 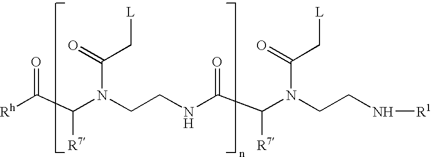
each L is independently selected from the group consisting of the nucleobases, in some embodiments, L is thymine, adenine, cytosine, guanine, or uracil;
each R⁷′ is hydrogen; and
n is an integer from 1 to 30.

Additional PNA compositions that are useful in the instant invention can be found in U.S. Pat. No. 6,326,479, which is incorporated by reference in its entirety. In some embodiments, the PNA is of the formula: 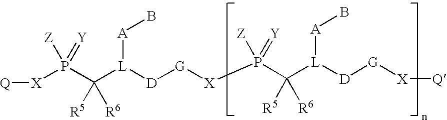
in which n is a number from zero to 100; A independently of one another is a single bond, a methylene group or a group of formula 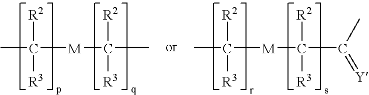
in which M is a single bond, —O—, —S— or —NR¹—, where R¹ is hydrogen or (C₁-C₆)-alkyl optionally substituted by hydroxyl, (C₁-C₆)-alkoxy, (C₁-C₆)-alkylthio or amino;
R² and R³ independently of one another are hydrogen, hydroxyl, (C₁-C₆)-alkoxy, (C₁-C₆)-alkylthio, amino, halogen, or (C₁-C₆)-alkyl optionally substituted by hydroxyl, (C₁-C₆)-alkoxy or (C₁-C₆)-alkylthio;
p and q independently of one another are zero to 5; and
r and s independently of one another are zero to 5;
B independently of one another is hydrogen, hydroxyl, (C₁-C₂₀)-alkyl, (C₁-C₂₀)-alkoxy, (C₁-C₂₀)-alkylthio, (C₆-C₂₀)-aryl-(C₁-C₆)-alkyl, (C₆-C₂₀)-aryl-(C₁-C₆)-alkoxy, (C₆-C₂₀)-aryl-(C₁-C₆)-alkythio, an aromatic group or a heterocyclic group, wherein the alkyl, alkyl portion of alkoxy or alkylthio, aromatic or heterocyclic group is optionally substituted one or more times by hydroxyl, (C₁-C₄)-alkoxy, —NR⁹R¹⁰, oxo, —C(O)OR⁸, —C(O)NR⁹R¹⁰, —CN, —F, —Cl, —Br, —NO₂, (C₂-C₆)-alkoxyalkyl, —S(O)ₘR⁸, —(C₁-C₆)-alkyl-S(O)ₘR⁸, —NHC(═NH)NHR⁸, —C(═NH)NHR⁹, NR⁹C(═O)R⁸, ═NOR⁸, NR⁹C(═O)OR¹⁰, —OC(═O)NR⁹R¹⁰, —NR⁹C(═O)NR⁹R¹⁰, a natural nucleobase, an unnatural nucleobase or a reporter ligand, with the proviso that at least one B moiety is a nucleobase;
m is zero, 1 or 2; or,
A-B independent of other A and B groups, can be a D- or L-amino acid condensed on via the carboxyl group or a peptide containing amino acids having a length of up to 5 amino acid residues, with the proviso that at least one B moiety is a nucleobase;
L independently of one another is N or R¹N⁺, where R¹ is as defined above; and
Y′ is ═O, ═S, ═CH₂, ═C(CH₃)₂ or ═NR¹, where R¹ is as defined above;
D and G each independently represent CR⁵R⁶ which can be the same or different;
R⁵ and R⁶ independently of one another are hydrogen, (C₁-C₆)-alkyl, (C₆-C₂₀)-aryl, (C₆-C₂₀)-aryl-(C₁-C₆)-alkyl, hydroxyl, (C₁-C₆)-alkoxy, (C₁-C₆)-alkylthio, wherein the alkyl, alkyl portion of alkoxy or alkylthio, or aryl group is optionally substituted by SR¹ or NR¹R¹, where R¹ is as defined above and R¹ independently of R¹ has the same meaning as R¹;
X independently of one another is —O—, —S— or —NR¹—, in which R¹ is as defined above;
Y independently of one another is ═O or ═S;
Z independently of one another is —OR⁸, —NR⁹R¹⁰ or X′Q″, where X′ is defined as X above and Q″ is defined as Q below;
R⁸ is hydrogen, (C₁-C₁₈)-alkyl, (C₂-C₁₈)-alkenyl, (C₃-C₁₈)-alkynyl, (C₆-C₁₂)-aryl, (C₆-C₁₂)-aryl-(C₁-C₆)-alkyl, wherein alkyl is optionally substituted one or more times by hydroxyl, (C₁-C₄)-alkoxy, F, Cl or Br and wherein aryl is optionally substituted 1-3 times by hydroxyl, (C₁-C₄)-alkoxy, (C₁-C₄)-alkyl, F, Cl, Br, NO₂, —NR⁹R¹⁰, —C(O)OH, —C(O)O—(C₁-C₆)-alkyl or —C(O)NR⁹R¹⁰;
R⁹ and R¹⁰ independently of one another are hydrogen, (C₁-C₁₈)-alkyl, (C₂-C₁₈)-alkenyl, (C₃-C₁₈)-alkynyl, (C₆-C₁₂)-aryl, (C₆-C₁₂)-aryl-(C₁-C₆)-alkyl, where alkyl is optionally substituted one or more times by hydroxyl, (C₁-C₄)-alkoxy, F, Cl or Br; or R⁹ and R¹⁰ form a 4 to 7-membered ring together with the N atom in —NR⁹R¹⁰;
Q and Q′ independently of one another are R⁸, modified or unmodified oligonucleotides or conjugates which a) favorably affect the properties of antisense oligonucleotides, b) affect the properties of triple helix-forming oligonucleotides, c) serve as a label of a DNA probe, or d) during the hybridization of the oligonucleotide analog to the target nucleic acid, attack the target nucleic acid with binding or cross-linking; or Q and Q′ alone or together are a single bond in a cyclic molecule; or Q and Q′, when neither is hydrogen, can be linked together to form a cyclic molecule.

These compositions can be synthesized by the methods of U.S. Pat. Nos. 5,874,533 and 6,127,346, the disclosures of which are incorporated in their entirety.

Yet other PNA compositions are of the formula: 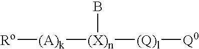
in which B—X is 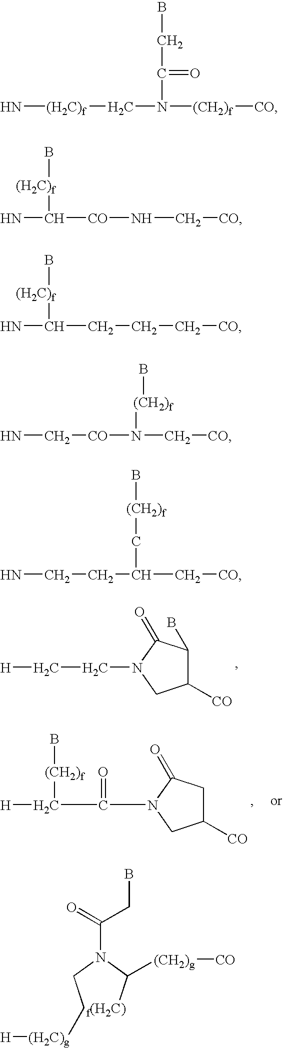
where f is 1-4 and g is 0-3; R^{o} is hydrogen, C₁-C₁₈-alkanoyl, C₁-C₁₈-alkoxy-carbonyl, C₃-C₈-cycloalkanoyl, C₇-C₁₅-aroyl, C₃-C₁₃-heteroaroyl, or a group which favors intracellular uptake of the oligomer;
A is an amino acid radical;
k is an integer from zero to 10;
Q is an amino acid radical;
l is an integer from zero to 10;
B is a natural nucleotide base or unnatural nucleotide base conventionally used in nucleotide chemistry or their prodrug forms, or a base substitute compound;
Q^{o} is hydroxyl, NH₂ or NHR″, in which R″ is C₁-C₁₈-alkyl, C₂-C₁₈-aminoalkyl or C₂-C₁₈-hydroxyalkyl; and
n is an integer from 1-50.

These compositions can be synthesized by the methods of U.S. Pat. No. 6,046,306, the disclosure of which are incorporated in its entirety.

Other PNA compositions are of the formula: 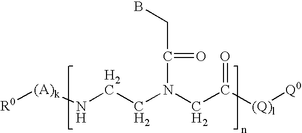
where R^{o} is hydrogen, C₁-C₁₈-alkanoyl, C₁-C₁₈-alkoxycarbonyl, C₃-C₈-cycloalkanoyl, C₇-C₁₅-aroyl, C₃-C₁₃-heteroaroyl, or a group which favors intracellular uptake of the oligomer;
A is an amino acid residue;
k is an integer from zero to 10;
Q is an amino acid residue;
m is an integer from 0 to 20;
B is a nucleotide base;
Q^{o} is hydroxyl, NH₂ or NHR″, with R″ is C₁-C₁₈-alkyl, C₂-C₁₈-aminoalkyl or C₂-C₁₈-hydroxyalkyl; and
n is an integer of 1-50;

These compounds may be made by methods disclosed in U.S. Pat. Nos. 6,121,418, 6,075,143, and 5,817,811, the disclosures of which are incorporated herein in their entirety by reference.

In certain embodiments, the PNA if of the formula:
-LQM-
wherein
Q is a linker or chemical bond;
one of L and M is a nucleotide moiety of the formula: 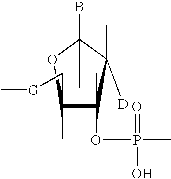
where B is a natural or unnatural nucleobase comprising a bond linking a nucleobase protecting group to the natural or unnatural nucleobase; and
D is a hydrogen atom, a hydroxyl group, a methoxyl group or a hydroxyl group which is protected by a protecting group;
the other of L and M is a PNA moiety of the formula: 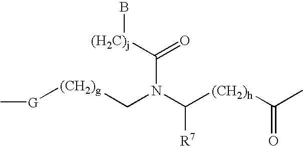
wherein G is a secondary nitrogen atom, a tertiary nitrogen atom having an alkyl substituent, an oxygen atom or a sulfur atom;
B is a natural or unnatural nucleobase;
R⁷ is selected from the group consisting of hydrogen and a side chain of a protected or unprotected naturally occurring α-amino acid; and
each of j, g and h is the same or different and is independently zero or an integer from one to five.

These compositions may be made by methods disclosed in U.S. Pat. No. 6,063,569, the disclosure of which is incorporated herein in its entirety.

Some PNA compositions may be of the formula: 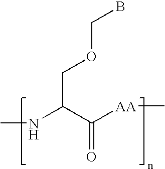
where B is a nucleobase, AA is an amino acid, and n is an integer greater than 1. Preferably, AA is an α-amino acid. These compositions can be synthesized by the method disclosed in U.S. Pat. No. 5,731,416, the disclosure of which is incorporated in its entirety.

In some embodiments, the oligomers are PNA mimic compositions. In certain embodiments, the PNA mimic composition is of the formula:
X—(B-L)ₙ-B—Y
where B is a nucleobase capable of effecting Watson/Crick base-pairing and bearing two linking attachement sites; L is a linker comprising 4-7 bonds; X and Y are terminating groups; and n is an integer equal to 1 or greater. Such compositions are synthesized by methods taught by U.S. Pat. No. 6,054,568, the disclosure of which is incorporated herein by reference in its entirety.

In certain embodiments, B is 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6diamino-7-deaza purine, 5-methyl-cytosine, 5-propynyl-uridine, 5-propynyl-cytidine, 2-thio-thymine or 2-thio-uridine

The term “terminating group” refers to one or more atoms located at the termini of the nucleobase oligomer. Terminating groups may possess functionality for chemical reactions to join other molecules or to effect changes in other molecules. Terminating groups may include capture labels, detection labels, and nucleic acids, including nucleic acid analogs. In some embodiments, X and Y are independently hydrogen, mono-substituted alkyl, di-substituted alkyl, methylene, mono-substituted methylene, alkylene, mono-substituted alkylene, aryl, substituted aryl, reactive functionality, detection labels, capture labels, DNA, RNA, or nucleic acid analogs.

Linkers, L, serve to attach the nucleobase monomers together to form the nucleobase oligomer. They are comprised of functionality that enables efficient and low-cost synthesis of the oligomer compounds and monomeric units. The present invention allows the use of a large variety of linker constructions. Preferred linkers are comprised of functionalities that enable efficient, automated, high-yield coupling reactions in the synthesis of nucleobase oligomers. The linkers may be used to confer nuclease resistance. The linkers determine the preferred conformation of the nucleobases, affecting affinity and specificity of base-pairing in hybridization. In some embodiments, L is methylene, alkylene, substituted alkylene, substituted aryl, phosphodiester, phosphotriester, alkylphosphonate, phosphoramidate, phosphorothioate, disulfide, amide, ester, carbonyl, sulfonamide, carbamate, urea, ethyleneoxy, reactive functionality, detection labels, or capture labels.

Morpholino nucleic acids are based on linked morpholino units having heterocyclic bases attached to the morpholino ring. Morpholino-based oligomeric compounds are disclosed, for example, U.S. Pat. No. 5,034,506, which is incorporated herein by reference. In some embodiments, morpholino nucleic acids comprise one or more monomers of the formula: 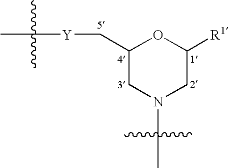
where
Y is a linking group; and
R^{1′} is hydrogen, hydroxy, (C₁-C₄)alkanoyl, naturally occurring nucleobases, non-naturally occurring nucleobases, aromatic moieties, DNA intercalators, nucleobase-binding groups, and heterocyclic moieties, reporter ligands. In preferred embodiments, at least one R^{1′} is a naturally occurring nucleobases or non-naturally occurring nucleobase. [0118] Y preferably is a one of the following types of linkages: phosphate, phosphorothioate; phosphorodithioate; phosphonate; phosphonothioate; phosphotriester; phosphorothiotriester; phosphoramidate; phosphorothioamidate; phosphinate; and boronate. Representative linkages also include ether-, allyl ether-, allyl sulfide-, formacetal/ketal- sulfide-, sulfoxide-, sulfone-, sulfamate-, sulfonamide-, siloxane-, amide-, cationic alkylpolyamine-, guanidyl-, morpholino-, hexose sugar and amide-containing linkages, and two to four atom linkages containing C, N, O, or S atoms. Preferred linkages are those that produce non-ionic oligomeric compound. In certain preferred embodiments, the intersubunit linkage group is: —NHC(═O)—O—, —CH₂CH₂—O—, —CH₂C(═O)—NH—, —SO₂—N(CH₃)—, N-alkylphoramidite, phosphothioate, or phosphate. It should be noted that the first four groups in the previous sentence can be linked at the morpholino nitrogen at either the right or left end of the depicted structure.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred linkages that do not contain a phosphorous atom include the following.

Ether. Certain embodiments of the invention relate to oligonucleotides containing at least one ether internucleoside linkage as described, for example, in U.S. Pat. No. 5,223,618, incorporated herein by reference in its entirety. In accordance with certain aspects of the invention, the ether linkage is of the formula —O—R₁—O— where R₁ is a group comprising a two or three carbon backbone. The two or three carbon component of the ether linkage can be widely functionalized. Thus, the group can be ethyl, ethylene, acetylene, cyclopropyl, cyclobutyl, ethylenoxy, ethylaziridine or substituted aziridine. Other cyclic structures can also be employed including propyl, isopropyl, methyl-cyclopropyl, C3 through C6 carbocyclic, and 4-, 5-, and 6-membered nitrogen heterocyclic moieties. The two or three carbon linking moiety and the cyclic structures can be widely substituted with amino, hydroxyl, carboxylic acids, and other groups that may enhance oligonucleotide properties such as solubility for formulations and cellular penetration.

Allyl ether and allyl sulfide. In certain embodiments, the invention relates to oligonucleotides containing allyl ether and allyl sulfide internucleoside linkages as described, for example, in U.S. Pat. No. 5,434,257, incorporated herein by reference in its entirety. The linkages comprise a three atom linkage of the formula 2′/3′-S—CH₂—CH=5′ or 2′/3′-O—CH₂CH=5′ wherein a double bond is located between the 5′ carbon atom and the adjacent substitute linkage atom (i.e., the carbon atom of —CH═).

Formacetal/ketal. In certain embodiments, the invention is directed to oligonucleotides that contain at least one formacetal/ketal type internucleoside linkage of the formula —YCX₂Y— wherein each Y is independently O or S and wherein each X is attached to the same carbon atom and provides sufficient electron withdrawal to stabilize the linkage. Such linkages are described, for example, in U.S. Pat. Nos. 5,264,562 and 5,264,564, hereby incorporated herein by reference in their entireties. In certain preferred embodiments, X is —H, —F, —Cl, —Br, —NO₂, —SCH₂CH₃, —COOH, —COOCH₃, —COOCH(CH₃)₂, —CONHCH₃, —CH₂F, —CF₃, —CH₂COOCH₃, —CH₂CONHCH₂CH₃, —CH₂CH₂COOH, —CH₂CH₂NH₂, —CH₂CH₂NHCH₂CH₃, or —CH₂CH₂CF₃. Particularly preferred are embodiments wherein both X are H or F or where both X taken together are —(CH₂)₂O(CH₂)₂.

Sulfide, Sulfoxide, and Sulfone. In certain aspects, the invention relates to oligonucleotides containing at least one internucleoside linking group comprising a sulfide (compounds with the general formula —S—) or sulfoxide (compounds with the general formula —SO—) as described, for example, in U.S. Pat. No. 5,216,141, hereby incorporated by reference in its entirety. In certain other embodiments, the invention relates to oligonucleotides containing at least one internucleoside linking group comprising a sulfone (compounds with the general formula —SO₂—) as described, for example, in U.S. Pat. Nos. 5,216,141 and 6,117,988, hereby incorporated by reference in their entireties.

Sulfamate. In certain other embodiments, the invention relates to oligonucleotides containing at least one sulfamate linkage of the following formula as described, for example, in U.S. Pat. No. 5,470,967, hereby incorporated by reference in its entirety: 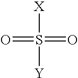
wherein X and Y are H or alkyl.

Sulfonamide. According to certain aspects of the present invention, oligonucleotides containing at least one sulfonate or sulfonamide internucleoside linkage are provided as described, for example, in U.S. Pat. No. 5,561,225, hereby incorporated by reference in its entirety. Specifically, internucleoside linkages of the following formulas are provided:
3′-B—SO₂—CH₂-5′ or 3′-B—SO₂—CH=5′
wherein B is oxygen, NHR³ with R₃ being hydrogen, C₁₋₅ alkyl optionally substituted by amino or hydroxy, piperidinyl, piperazinyl, morpholinyl, phenyl, benzyl, allyl, acetyl, or benzoyl.

Siloxane. Certain embodiments of the invention relate to oligonucleotides having at least one siloxane internucleoside linkage as described, for example, in U.S. Pat. No. 5,214,134, hereby incorporated herein by reference in its entirety. The siloxane internucleoside linkage has the following formula: 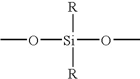
wherein each R is independently C1-C6 alkyl. In a preferred embodiment, R is methyl or isopropyl.

Amide. In certain aspects of the invention, oligonucleotides are provided that contain at least one amide internucleoside linkage as described, for example, in U.S. Pat. Nos. 5,602,240 and 5,663,312, hereby incorporated by reference in their entireties. In certain embodiments of the invention, the amide linkages are NR—C(O)—CH₂—CH₂, NR—C(S)—CH₂—CH₂, CH₂—NR—C(O)—CH₂, CH₂—NR—C(S)—CH₂, CH₂—CH₂—NR—C(O), CH₂—CH₂—NR—C(S), C(O)—NR—CH₂—CH₂, C(S)—NR—CH₂CH₂, CH₂—C(O)—NR—CH₂, or CH₂—C(S)—NR—CH₂ where R is hydrogen, alkyl, substituted alkyl, aralkyl, alkenyl, alkaryl, aminoalkyl, hydroxyalkyl, heterocycloalkyl, heterocycloaralkyl, an RNA cleaving group, a group for improving the affinity for the RNA complement or a group for improving the pharmacodynamic properties of the oligonucleotide. In other embodiments, the amide linkages are -3′-(CR₃R₄)ₘ—CONR₁—(CR₃R⁴)ₙ-5′ - or -3′-(OR₃R⁴)ₘ—NR₁CO—(CR₃R⁴)ₙ-5′-wherein each R₁, R₃, and R₄ is selected from the group consisting of H, lower alkyl, aryl, aryl-lower alkyl, and lower alkenyl; and m and n are independently 0, 1 or 2.

Cationic alkylpolyamine linkages. In one aspect of the invention, the invention relates to oligonucleotides that contain cationic alkylpolyamine internucleoside linkages as described, for example, in U.S. Pat. No. 6,331,617, hereby incorporated by reference in its entirety. In a preferred embodiment, the oligonucleotide comprises dimethylamino propylamine linkages, N,N-diaminopropylamine linkages, or diethyethylinediamine linkages. In another embodiment, the oligonucleotide comprises ethylenediamine linkages and mixed chirality dimethylamino propylamine linkages or diethylethylenediamine linkages.

Positively charged guanidyl linkages. In certain embodiments, the invention relates to oligonucleotides that contain at least one positively charged guanidyl internucleoside linkage as described, for example, in U.S. Pat. No. 6,013,785, incorporated herein by reference in its entirety. In preferred embodiments, the positively charged guanidyl linkage has the following formula: 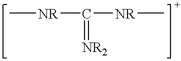
wherein R is a hydrogen molecule or a lower alkyl or phenyl group that does not alter the positive charge on guanidine. Examples of suitable lower alkyl groups include, but are not limited to, CH₃, CH₂CH₃, CH₃(CH₂)ₙCH₃, CH₂X, CH₃CH₂X, CH₃(CH₂)ₙCH₂X, wherein X is any functional group that does not alter the positive charge on guanidine and n is 0, 1, or 2. Examples include, but are not limited to, —OH or —NH₂.

Two to four atom 2′-5′ linking group. In certain aspects, the invention relates to oligonucleotides that contain at least one two to four atom 2′-5′ internucleoside linkage wherein at least one of the atoms making up the internucleoside linkage is selected from nitrogen, oxygen or sulfur, with the remainder being carbon, as described, for example, in U.S. Pat. Nos. 5,817,781 and 6,410,702, hereby incorporated herein by reference in their entireties. In preferred embodiments, such linkages are: —S—CH₂—CH₂—, —S—CH₂—, —O—CH₂—S—, —O—CH₂—O—, —CH₂—CH₂—S—, —CH₂—S—, —S—CH₂—O—, 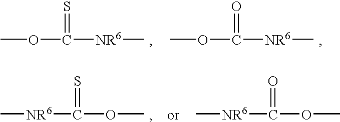
wherein R⁶ is lower alkyl (including methyl, ethyl, propyl, and butyl), OMe, OH, heteroalkyl, or aryl.

Two to four atom linkage containing at least one N, O, or S atom with the remainder being C atoms. In certain embodiments, the present invention is directed to oligonucleotides containing at least one internucleoside linkage that is a two to four atom internucleoside linkage wherein at least one of the atoms making up the internucleoside linkage is selected from nitrogen, oxygen and sulfur, with the remainder being carbon. Such modified oligonucleotides are described, for example, in U.S. Pat. No. 5,596,086, hereby incorporated by reference in its entirety. Particularly preferred of such internucleoside linkages include —CH₂—CH₂—NR—, —NR—CH₂—CH₂—, —CH₂—NR—CH₂—, —CH₂CH₂—CH₂—O—, —CH₂—O—CH₂—, —S—CH₂—CH₂—, and —O—CH₂—CH₂—NR— wherein R is hydrogen, lower alkyl, heteroalkyl, aryl, sulfonamide, phosphoramidate, NR′, OR′, 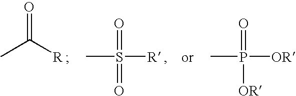
and R′ is hydrogen, lower alkyl, heteroalkyl, or aryl.

Three atom linking groups. In certain embodiments, the invention relates to oligonucleotides containing at least one three atom internucleoside linkage of the following formula as described, for example, in U.S. Pat. No. 5,677,439, hereby incorporated by reference in its entirety:
-D-D-D-
where each D is independently CHR, oxygen or NR₆, wherein R is hydrogen, OH, SH or NH₂, R₆ is hydrogen or C₁-C₂ alkyl, and only one D is oxygen or NR₆.

Four-atom linking group containing at least two carbon atoms. In other embodiments, the invention relates to oligonucleotides containing at least one internucleoside linkage comprised of a four atom linking group as described, for example, in U.S. Pat. No. 5,610,289, hereby incorporated by reference in its entirety. Included within the four atom linker is preferably a 3′-deoxy function on one of the linked sugars. The four atom linker is of the structure -L₁-L₂-L₃-L₄- wherein L₁ and L₄ are methylene carbon atoms or substituted carbon atoms and L₂ and L₃ are methylene carbon atoms, substituted carbon atoms, oxygen atoms, nitrogen or substituted nitrogen atoms, substituted phosphorus atoms, sulfur or substituted sulfur atoms or substituted silicon atoms. The linkage may be neutral or may be positively or negatively charged.

Four atom linking group containing at least two carbon atoms with the remainder of the atoms selected from optionally substituted C, P, O, S, and N atoms. In certain embodiments, the invention relates to oligonucleotides that contain at least one internucleoside linkage of formula L₁-L₂-L₃-L₄ as described, for example, in U.S. Pat. No. 5,965,721, hereby incorporated by reference in its entirety, wherein L₁ and L₄ are, independently, CH₂, C═O, C═S, C—NH₂, C—NHR₃, C—OH, C—SH, C—O—R₁ or C—S—R₁. L₂ and L₃ are, independently, CR₁R₂, C═CR₁R₂, C═NR₃, P(O)R⁴, P(S)R⁴, C═O, C═S, O, S, SO, SO₂, NR³ or SiR₅R⁶; or together, form part of an alkene, alkyne, aromatic ring, carbocycle or heterocycle. L₁, L₂, L₃ and L₄ are as noted, but if L₁ is C═O or C═S then L₂ is not NR³ or if L₄ is C═O or C═S then L₃ is not NR³. Further L₁, L₂, L₃ and L₄, together, may comprise a —CH═N—NH—CH₂— or —CH₂—O—N═CH- moiety. R₁ and R₂ are, independently, H; OH; SH; NH₂; C₁ to C₁₀ alkyl, substituted alkyl, alkenyl, alkaryl or aralkyl; alkoxy; thioalkoxy; alkylamino; aralkylamino; substituted alkylamino; heterocycloalkyl; heterocycloalkylamino; aminoalkylamino; polyalkylamino; halo; formyl; keto; benzoxy; carboxamido; thiocarboxamido; ester; thioester; carboxamidine; carbamyl; ureido or guanidino. They may also independently comprise an RNA cleaving group; a group for improving the pharmacokinetic properties of an oligonucleotide; or a group for improving the pharmacodynamic properties of an oligonucleotide. R₃ is H, OH, NH₂, lower alkyl, substituted lower alkyl, alkoxy, lower alkenyl, aralkyl, alkylamino, aralkylamino, substituted alkylamino, heterocycloalkyl, heterocycloalkylamino, aminoalkylamino, polyalkylamino, an RNA cleaving group, a group for improving the pharmacokinetic properties of an oligonucleotide or a group for improving the pharmacodynamic properties of an oligonucleotide. R⁴ is OH, SH, NH₂, O-alkyl, S-alkyl, NH-alkyl, O-alkylheterocyclo, S-alkylheterocyclo, N-alkylheterocyclo or a nitrogen-containing heterocycle. R₅ and R₆ are, independently, C₁ to C₆ alkyl or alkoxy.

Four atom linking group containing adjacent N atoms or adjacent N and O atoms. In accordance with certain aspects of the invention, oligonucleotides are provided that contain at least one internucleoside linkage that is a four atom linking group as described, for example, in U.S. Pat. Nos. 5,489,677, 5,541,307, 5,618,704, 5,808,023, 5,965,721, 5,969,118, and 6,420,549, hereby incorporated by reference in their entireties. The four atom linking group is of the following formulas: CH₂—R_{A}—NR—CH₂, CH₂—NR—R_{A}—CH₂, R_{A}—NR—CH₂— CH₂, CH₂—CH₂—NR—R_{A}, CH₂—CH₂—R_{A}—NR, NR—R_{A}—CH₂—CH₂, or NR—R_{A}—CH₂ where R_{A} is O or NR and R is H; alkyl or substituted alkyl having 1 to about 10 carbon atoms; alkenyl or substituted alkenyl having 2 to about 10 carbon atoms; alkynyl or substituted alkynyl having 2 to about 10 carbon atoms; alkaryl, substituted alkaryl, aralkyl, or substituted aralkyl having 7 to about 14 carbon atoms; alicyclic; heterocyclic; a reporter molecule; an RNA cleaving group; a group for improving the pharmacokinetic properties of an oligonucleotide; or a group for improving the pharmacodynamic properties of an oligonucleotide. In certain preferred embodiments, L₁-L₂-L₃-L₄ is CH₂—O—NR—CH₂, CH₂—NR—O—CH₂, O—NR—CH₂—CH₂, or NR—O—CH₂—CH₂. In other preferred embodiments L₁-L₂-L₃-L₄ is a CH₂—N(CH₃)—O—CH₂ linkage. The CH₂—N(CH₃)—O—CH₂ linkage can be named in various ways, including a 3′-de(oxyphosphinico)-3′-[methylene (methylimino)] linkage, which can be shorten to a methylene(methylimino) linkage or shortened still to the acronym “MMI” linkage.

Four atom linking groups containing adjacent nitrogen atoms. In further embodiments, the invention relates to oligonucleotides that contain at least one internucleoside linkage that contains adjacent nitrogen atoms as described, for example, in U.S. Pat. No. 5,792,844, incorporated herein by reference in its entirety. In preferred embodiments, the internucleoside linkage is —CH₂—NR₁— NR₂—CH₂— or —NR₁—NR₂—CH₂—CH₂— wherein R₁ and R₂ are the same or different and are H; alkyl or substituted alkyl having 1 to about 10 carbon atoms; alkenyl or substituted alkenyl having 2 to about 10 carbon atoms; alkynyl or substituted alkynyl having 2 to about 10 carbon atoms; alkaryl, substituted alkaryl, aralkyl, or substituted aralkyl having 7 to about 14 carbon atoms; alicyclic; heterocyclic; a reporter molecule; an RNA cleaving group; a group for improving the pharmacokinetic properties of an oligonucleotide; or a group for improving the pharmacodynamic properties of an oligonucleotide.

Four atom linking group containing at least one O, S, or N atom. In certain embodiments, the invention relates to oligonucleotides that contain at least one internucleoside linkage of formula L₁-L₂-L₃-L₄ as described, for example, in U.S. Pat. No. 5,777,092, hereby incorporated by reference in its entirety, wherein one of L₁ or L₂ is O or S, and the other of L₁ or L₂ is N—R, and combined L₃ and L₄ are CH₂, or L₃ is CH₂ and L₄ is CR′R″; or one of L₃ or L₄ is O or S, and the other of L₃ or L₄ is N—R, and combined L₁ and L₂ are CH₂, or L₂ is CH₂ and L₁ is CR′R″; or one of L₁ and L₄ is O, S or N—R, and the other of L₁ and L₄ is CR′R″, and L₂ and L₃ are CH₂; or L₁, L₂, L₃ and L₄, together, are O—N═CH— CH₂ or CH₂—CH═N—O; or L₁ is O, L₂ is N, L₃ is CH₂, and L₄ is C or CH, and together with at least two additional carbon or hetero atoms, L₂, L₃ and L₄ form a 5 or 6 membered ring; or L₁ is C or CH, L₂ is CH₂, L₃ is N, and L₄ is O, and together with at least two additional carbon or hetero atoms, L₁, L₂ and L₃ form a 5 or 6 membered ring; and R is H; C₁ to C₁₀ straight or branched chain lower alkyl or substituted lower alkyl; C₂ to C₁₀ straight or branched chain lower alkenyl or substituted lower alkenyl; C₂ to C₁₀ straight or branched chain lower alkynyl or substituted lower alkynyl; a ¹⁴C containing lower alkyl, lower alkenyl or lower alkynyl; C₇ to C₁₄ substituted or unsubstituted alkaryl or aralkyl; a ¹⁴C containing C₇ to C₁₄ alkaryl or aralkyl; alicyclic; heterocyclic; a reporter molecule; an RNA cleaving group; a group for improving the pharmacokinetic properties of an oligonucleotide; or a group for improving the pharmacodynamic properties of an oligonucleotide; R′ and R″ are H; or R′ is H and R″ is O—R; or combined R′ and R″ are ═O.

Four atom linking group containing at least one N, S, Se, P, Si, or O atom. In certain embodiments, the invention relates to oligonucleotides that contain at least one internucleoside linkage of formula L₁-L₂-L₃-L₄ as described, for example, in U.S. Pat. No. 5,677,437, hereby incorporated by reference in its entirety, wherein L₁-L₂-L₃-L₄ is CR₁ₐR_{1b}—CR₂ₐR_{2b}—CR₃ₐR_{3b}-Z₄, CR₁ₐR_{1b}—CR₂ₐR_{2b}-Z₃-Z₄, CR₁ₐR_{1b}-Z₂-CR₂ₐR_{2b}-Z₄, Z₁-CR₁ₐR_{1b}-CR₂ₐR_{2b}-Z₄, CR₁ₐR_{1b}-Z₂-Z₃-Z₄, Z₁-CR^{2a}R^{2b}-Z₃-Z₄, or Z₁-Z₂-CR^{3a}R^{3b}-Z₄ wherein Z₁, Z₂, Z₃ and Z₄ are, independently, NR₄, S, SO, SO₂, Se, P(=J₁)J₂, Si(R₆)₂, or O; R₁ₐ, R_{1b}, R₂ₐ, R_{2b}, R₃ₐ and R^{3b} are, independently, H, R₅, O—R₅, S—R₅, NR⁴R⁵; or, independently, together R₁ₐ and R_{1b}, or R^{2a} and R^{2b}, or R^{3a} and R^{3b} are ═O; J₁ is O, S, Se, or NR⁴; J₂ is OH, OR₅, SH, SR₅, SeH, R₅, BH₃ or NR⁴R₅; and R⁴, R₅ and R₆ are, independently, H; C₁ to C₁₀ straight or branched chain lower alkyl or substituted lower alkyl; C₂ to C₁₀ straight or branched chain lower alkenyl or substituted lower alkenyl; C₂ to C₁₀ straight or branched chain lower alkynyl or substituted lower alkynyl; a ¹⁴C containing lower alkyl, lower alkenyl or lower alkynyl; C₇ to C₁₄ substituted or unsubstituted alkaryl or aralkyl; a ¹⁴C containing C₇ to C₁₄ alkaryl or aralkyl; C₆ to C₁₄ aryl; alicyclic; heterocyclic; a reporter molecule; an RNA cleaving group; a group for improving the pharmacokinetic properties of an oligonucleotide; or a group for improving the pharmacodynamic properties of an oligonucleotide; and where said substituents are OH, ═O, CO₂H, O-alkyl, SH, S-alkyl, NH-alkyl, N-(alkyl)₂, alkyl, F, Cl, Br, CN, CF₃, OCF₃, OCN, SOCH₃, SO₂CH₃, ONO₂, NO₂, N₃, NH₂, heterocycloalkyl, aryl, aralkyl, sulfide, silyl, intercalators, conjugates, imidazoles, amides, ester, ethers, carbonates, carbamates, ureas, polyamines, polyamides, polyethylene glycols or polyethers.

Further teachings and methods for preparation of morpholino nucleic acids can be found in U.S. Pat. Nos. 5,405,938, 5,235,033, 5,188,879, and 5,166,315.

In certain embodiments, the invention is directed to oligonucleotides that contain at least one internucleoside linkage comprised of a hexose sugar and an amide as described, for example, in U.S. Pat. No. 5,780,607, hereby incorporated by reference in its entirety. In certain preferred embodiments, such oligonucleotides have the following formula: 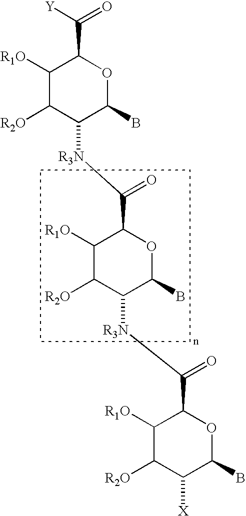
wherein R₁ and R² are independently hydrogen, lower alkyl or acyl; R³ is hydrogen or lower alkyl; B is a nucleobase or a protected nucleobase, such that said oligomer has a sequence of bases complementary to a selected RNA sequence; n is 5 to 30; X is NR³R⁴; and Y is OR³, or NHR³.

Cyclohexenyl nucleic acids (CeNA) are compositions where the furanose ring normally present in an DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligomers having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. Coli RNase resulting in cleavage of the target RNA strand.

The general formula of CeNA is shown below: 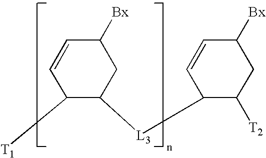
wherein
each Bx is a heterocyclic base moiety;
T₁ is hydroxyl or a protected hydroxyl; and
T2 is hydroxyl or a protected hydroxyl.

Acyclic backbone moieties according to the invention have one of the formulas: 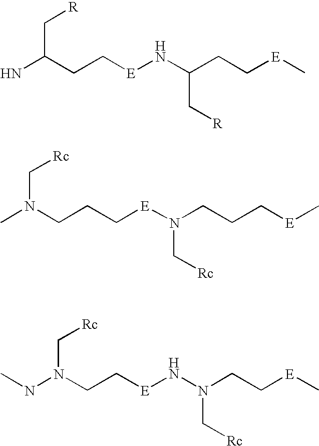
where E is C(═O) or SO₂; and
R and Rc are each independently selected from the group consisting of hydrogen, hydroxy, (C₁-C₄)alkanoyl, naturally occurring nucleobases, non-naturally occurring nucleobases, aromatic moieties, DNA intercalators, nucleobase-binding groups, heterocyclic moieties, and reporter ligands, at least one of R and Rc being a naturally occurring nucleobase, a non-naturally occurring nucleobase, a DNA intercalator, or a nucleobase-binding group. In some embodiments, at least one R or Rc is a naturally occurring nucleobase, a non-naturally occurring nucleobase.

These compositions can be prepared by the methods presented in PCT Patent Application No. WO 86/05518.

Hybridization

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases that pair through the formation of hydrogen bonds. Hybridization can occur under varying circumstances.

An oligomeric compound of the invention is believed to specifically hybridize to the target nucleic acid and interfere with its normal function to cause a loss of activity. There is preferably a sufficient degree of complementarity to avoid non-specific binding of the oligomeric compound to non-target nucleic acid sequences under conditions in which specific binding is desired, i.e., under physiological conditions in the case of in vivo assays or therapeutic treatment, and under conditions in which assays are performed in the case of in vitro assays.

In the context of the present invention the phrase “stringent hybridization conditions” or “stringent conditions” refers to conditions under which an oligomeric compound of the invention will hybridize to its target sequence, but to a minimal number of other sequences. Stringent conditions are sequence-dependent and will vary with different circumstances and in the context of this invention; “stringent conditions” under which oligomeric compounds hybridize to a target sequence are determined by the nature and composition of the oligomeric compounds and the assays in which they are being investigated.

“Complementary,” as used herein, refers to the capacity for precise pairing of two nucleobases regardless of where the two are located. For example, if a nucleobase at a certain position of an oligomeric compound is capable of hydrogen bonding with a nucleobase at a certain position of a target nucleic acid, then the position of hydrogen bonding between the oligomer and the target nucleic acid is considered to be a complementary position. The oligomeric compound and the target nucleic acid are complementary to each other when a sufficient number of complementary positions in each molecule are occupied by nucleobases that can hydrogen bond with each other. Thus, “specifically hybridizable” and “complementary” are terms which are used to indicate a sufficient degree of precise pairing or complementarity over a sufficient number of nucleobases such that stable and specific binding occurs between the oligomer and a target nucleic acid.

It is understood in the art that the sequence of the oligomeric compound need not be 100% complementary to that of its target nucleic acid to be specifically hybridizable. Moreover, an oligomeric compound may hybridize over one or more segments such that intervening or adjacent segments are not involved in the hybridization event (e.g., a loop structure or hairpin structure). It is preferred that the oligomeric compounds of the present invention comprise at least 70% sequence complementarity to a target region within the target nucleic acid, more preferably that they comprise 90% sequence complementarity and even more preferably comprise 95% sequence complementarity to the target region within the target nucleic acid sequence to which they are targeted. For example, an oligomeric compound in which 18 of 20 nucleobases of the oligomeric compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Targets of the Invention

“Targeting” an oligomeric compound to a particular nucleic acid molecule, in the context of this invention, can be a multistep process. The process usually begins with the identification of a target nucleic acid whose function is to be modulated. This target nucleic acid may be, for example, a mRNA transcribed from a cellular gene whose expression is associated with a particular disorder or disease state, or a nucleic acid molecule from an infectious agent.

The targeting process usually also includes determination of at least one target region, segment, or site within the target nucleic acid for the interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid. The terms region, segment, and site can also be used to describe an oligomeric compound of the invention such as for example a gapped oligomeric compound having 3 separate segments.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding a nucleic acid target, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense oligomeric compounds of the present invention.

The open reading frame (ORF) or “coding region,” which is known in the art to refer to the region between the translation initiation codon and the translation termination codon, is also a region which may be targeted effectively. Within the context of the present invention, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using oligomeric compounds targeted to, for example, pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequences.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also preferred target nucleic acids.

The locations on the target nucleic acid to which preferred compounds and compositions of the invention hybridize are herein below referred to as “preferred target segments.” As used herein the term “preferred target segment” is defined as at least an 8-nucleobase portion of a target region to which an active antisense oligomeric compound is targeted. While not wishing to be bound by theory, it is presently believed that these target segments represent portions of the target nucleic acid that are accessible for hybridization.

Once one or more target regions, segments or sites have been identified, oligomeric compounds are chosen which are sufficiently complementary to the target, i.e., hybridize sufficiently well and with sufficient specificity, to give the desired effect.

In accordance with an embodiment of the this invention, a series of nucleic acid duplexes comprising the antisense strand oligomeric compounds of the present invention and their respective complement sense strand compounds can be designed for a specific target or targets. The ends of the strands may be modified by the addition of one or more natural or modified nucleobases to form an overhang. The sense strand of the duplex is designed and synthesized as the complement of the antisense strand and may also contain modifications or additions to either terminus. For example, in one embodiment, both strands of the duplex would be complementary over the central nucleobases, each having overhangs at one or both termini.

For the purposes of describing an embodiment of this invention, the combination of an antisense strand and a sense strand, each of can be of a specified length, for example from 18 to 29 nucleotides long, is identified as a complementary pair of siRNA oligomers. This complementary pair of siRNA oligomers can include additional nucleotides on either of their 5′ or 3′ ends. Further they can include other molecules or molecular structures on their 3′ or 5′ ends such as a phosphate group on the 5′ end. A preferred group of compounds of the invention include a phosphate group on the 5′ end of the antisense strand compound. Other preferred compounds also include a phosphate group on the 5′ end of the sense strand compound. An even further preferred compounds would include additional nucleotides such as a two base overhang on the 3′ end.

For example, a preferred siRNA complementary pair of oligomers comprise an antisense strand oligomeric compound having the sequence CGAGAGGCGGACGGGACCG (SEQ ID NO:1) and having a two-nucleobase overhang of deoxythymidine(dT) and its complement sense strand. These oligomers would have the following structure:

| | | |
|---|---|---|
| 5′ c g a g a g g c g g a c g g g a c c g T T 3′ | Antisense Strand | (SEQ ID NO:2) |
| \| \|\| \| \| \| \| \| \| \|\| \| \| \| \| \| \| \| \| | | |
| 3′ T T g c t c t c c g c c t g c c c t g g c 5′ | Compliment Strand | (SEQ ID NO:3) |

In an additional embodiment of the invention, a single oligomer having both the antisense portion as a first region in the oligomer and the sense portion as a second region in the oligomer is selected. The first and second regions are linked together by either a nucleotide linker (a string of one or more nucleotides that are linked together in a sequence) or by a non-nucleotide linker region or by a combination of both a nucleotide and non-nucleotide structure. In each of these structures, the oligomer, when folded back on itself, would be complementary at least between the first region, the antisense portion, and the second region, the sense portion. Thus the oligomer would have a palindrome within it structure wherein the first region, the antisense portion in the 5′ to 3′ direction, is complementary to the second region, the sense portion in the 3′ to 5′ direction.

In a further embodiment, the invention includes oligomer/protein compositions. Such compositions have both an oligomer component and a protein component. The oligomer component comprises at least one oligomer, either the antisense or the sense oligomer but preferably the antisense oligomer (the oligomer that is antisense to the target nucleic acid). The oligomer component can also comprise both the antisense and the sense strand oligomers. The protein component of the composition comprises at least one protein that forms a portion of the RNA-induced silencing complex, i.e., the RISC complex.

RISC is a ribonucleoprotein complex that contains an oligomer component and proteins of the Argonaute family of proteins, among others. While we do not wish to be bound by theory, the Argonaute proteins make up a highly conserved family whose members have been implicated in RNA interference and the regulation of related phenomena. Members of this family have been shown to possess the canonical PAZ and Piwi domains, thought to be a region of protein-protein interaction. Other proteins containing these domains have been shown to effect target cleavage, including the RNAse, Dicer. The Argonaute family of proteins includes, but depending on species, are not necessary limited to, elF2C1 and elF2C2. elF2C2 is also known as human GERp95. While we do not wish to be bound by theory, at least the antisense oligomer strand is bound to the protein component of the RISC complex. Additional, the complex might also include the sense strand oligomer. Carmell et al, Genes and Development 2002, 16, 2733-2742.

Also while we do not wish to be bound by theory, it is further believed that the RISC complex may interact with one or more of the translation machinery components. Translation machinery components include but are not limited to proteins that effect or aid in the translation of an RNA into protein including the ribosomes or polyribosome complex. Therefore, in a further embodiment of the invention, the oligomer component of the invention is associated with a RISC protein component and further associates with the translation machinery of a cell. Such interaction with the translation machinery of the cell would include interaction with structural and enzymatic proteins of the translation machinery including but not limited to the polyribosome and ribosomal subunits.

In a further embodiment of the invention, the oligomer of the invention is associated with cellular factors such as transporters or chaperones. These cellular factors can be protein, lipid or carbohydrate based and can have structural or enzymatic functions that may or may not require the complexation of one or more metal ions.

Furthermore, the oligomer of the invention itself may have one or more moieties which are bound to the oligomer which facilitate the active or passive transport, localization or compartmentalization of the oligomer. Cellular localization includes, but is not limited to, localization to within the nucleus, the nucleolus or the cytoplasm. Compartmentalization includes, but is not limited to, any directed movement of the oligomers of the invention to a cellular compartment including the nucleus, nucleolus, mitochondrion, or imbedding into a cellular membrane surrounding a compartment or the cell itself.

In a further embodiment of the invention, the oligomer of the invention is associated with cellular factors that affect gene expression, more specifically those involved in RNA modifications. These modifications include, but are not limited to posttrascriptional modifications such as methylation. Furthermore, the oligomer of the invention itself may have one or more moieties which are bound to the oligomer which facilitate the posttranscriptional modification.

The oligomeric compounds of the invention may be used in the form of single-stranded, double-stranded, circular or hairpin oligomeric compounds and may contain structural elements such as internal or terminal bulges or loops. Once introduced to a system, the oligomeric compounds of the invention may interact with or elicit the action of one or more enzymes or may interact with one or more structural proteins to effect modification of the target nucleic acid.

One non-limiting example of such an interaction is the RISC complex. Use of the RISC complex to effect cleavage of RNA targets thereby greatly enhances the efficiency of oligomer-mediated inhibition of gene expression. Similar roles have been postulated for other ribonucleases such as those in the RNase III and ribonuclease L family of enzymes.

Preferred forms of oligomeric compound of the invention include a single-stranded antisense oligomer that binds in a RISC complex, a double stranded antisense/sense pair of oligomer or a single strand oligomer that includes both an antisense portion and a sense portion. Each of these compounds or compositions is used to induce potent and specific modulation of gene function. Such specific modulation of gene function has been shown in many species by the introduction of double-stranded structures, such as double-stranded RNA (dsRNA) molecules and has been shown to induce potent and specific antisense-mediated reduction of the function of a gene or its associated gene products. This phenomenon occurs in both plants and animals and is believed to have an evolutionary connection to viral defense and transposon silencing.

The compounds and compositions of the invention are used to modulate the expression of a target nucleic acid. “Modulators” are those oligomeric compounds that decrease or increase the expression of a nucleic acid molecule encoding a target and which comprise at least an 8-nucleobase portion that is complementary to a preferred target segment. The screening method comprises the steps of contacting a preferred target segment of a nucleic acid molecule encoding a target with one or more candidate modulators, and selecting for one or more candidate modulators which decrease or increase the expression of a nucleic acid molecule encoding a target. Once it is shown that the candidate modulator or modulators are capable of modulating (e.g. either decreasing or increasing) the expression of a nucleic acid molecule encoding a target, the modulator may then be employed in further investigative studies of the function of a target, or for use as a research, diagnostic, or therapeutic agent in accordance with the present invention.

Oligomeric Compounds

In the context of the present invention, the term “oligomeric compound” refers to a polymeric structure capable of hybridizing a region of a nucleic acid molecule. This term includes oligonucleotides, oligonucleosides, oligonucleotide analogs, oligonucleotide mimetics and combinations of these. Oligomeric compounds routinely prepared linearly but can be joined or otherwise prepared to be circular and may also include branching. Oligomeric compounds can hybridized to form double stranded compounds that can be blunt ended or may include overhangs. In general an oligomeric compound comprises a backbone of linked momeric subunits where each linked momeric subunit is directly or indirectly attached to a heterocyclic base moiety. The linkages joining the monomeric subunits, the sugar moieties or surrogates and the heterocyclic base moieties can be independently modified giving rise to a plurality of motifs for the resulting oligomeric compounds including hemimers, gapmers and chimeras.

As is known in the art, a nucleoside is a base-sugar combination. The base portion of the nucleoside is normally a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligomers, the phosphate groups covalently link adjacent nucleosides to one another to form a linear polymeric compound. The respective ends of this linear polymeric structure can be joined to form a circular structure by hybridization or by formation of a covalent bond, however, open linear structures are generally preferred. Within the oligomer structure, the phosphate groups are commonly referred to as forming the internucleoside linkages of the oligomer. The normal internucleoside linkage of RNA and DNA is a 3′ to 5′ phosphodiester linkage.

In the context of this invention, the term “oligonucleotide” refers to an oligomer or polymer of ribonucleic acid (RNA) or deoxyribonucleic acid (DNA). This term includes oligonucleotides composed of naturally-occurring nucleobases, sugars and covalent internucleoside linkages. The term “oligonucleotide analog” refers to oligonucleotides that have one or more non-naturally occurring portions which function in a similar manner to oligonulceotides. Such non-naturally occurring oligonucleotides are often preferred the naturally occurring forms because of desirable properties such as, for example, enhanced cellular uptake, enhanced affinity for nucleic acid target and increased stability in the presence of nucleases.

In the context of this invention, the term “oligonucleoside” refers to nucleosides that are joined by internucleoside linkages that do not have phosphorus atoms. Internucleoside linkages of this type include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetal, formacetal, thioformacetal, methylene formacetal, thioformacetal, alkeneyl, sulfamate; methyleneimino, methylenehydrazino, sulfonate, sulfonamide, amide and others having mixed N, O, S and CH₂ component parts.

In addition to the modifications described above, the nucleosides of the oligomeric compounds of the invention can have a variety of other modification so long as these other modifications either alone or in combination with other nucleosides enhance one or more of the desired properties described above. Thus, for nucleotides that are incorporated into oligomers of the invention, these nucleotides can have sugar portions that correspond to naturally-occurring sugars or modified sugars. Representative modified sugars include carbocyclic or acyclic sugars, sugars having substituent groups at one or more of their 2′, 3′ or 4′ positions and sugars having substituents in place of one or more hydrogen atoms of the sugar. Additional nucleosides amenable to the present invention having altered base moieties and or altered sugar moieties are disclosed in U.S. Pat. No. 3,687,808 and PCT application PCT/US89/02323.

Altered base moieties or altered sugar moieties also include other modifications consistent with the spirit of this invention. Such oligomers are best described as being structurally distinguishable from, yet functionally interchangeable with, naturally occurring or synthetic wild type oligonucleotides. All such oligomers are comprehended by this invention so long as they function effectively to mimic the structure of a desired RNA or DNA strand. A class of representative base modifications include tricyclic cytosine analog, termed “G clamp” (Lin, et al., J. Am. Chem. Soc. 1998, 120, 8531). This analog makes four hydrogen bonds to a complementary guanine (G) within a helix by simultaneously recognizing the Watson-Crick and Hoogsteen faces of the targeted G. This G clamp modification when incorporated into phosphorothioate oligomers, dramatically enhances antisense potencies in cell culture. The oligomers of the invention also can include phenoxazine-substituted bases of the type disclosed by Flanagan, et al., Nat. Biotechnol. 1999, 17(1), 48-52.

The oligomeric compounds in accordance with this invention preferably comprise from about 8 to about 80 nucleobases (i.e. from about 8 to about 80 linked nucleosides). One of ordinary skill in the art will appreciate that the invention embodies oligomeric compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one preferred embodiment, the oligomeric compounds of the invention are 12 to 50 nucleobases in length. One having ordinary skill in the art will appreciate that this embodies oligomeric compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred embodiment, the oligomeric compounds of the invention are 15 to 30 nucleobases in length. One having ordinary skill in the art will appreciate that this embodies oligomeric compounds of 15, 16, 17, 18, 19, 20, 21, 22, 23, 24, 25, 26, 27, 28, 29, or 30 nucleobases in length.

Particularly preferred oligomeric compounds are oligomers from about 12 to about 50 nucleobases, even more preferably those comprising from about 15 to about 30 nucleobases.

General Oligomer Synthesis

Oligomerization of modified and unmodified nucleosides is performed according to literature procedures for DNA like compounds (Protocols for Oligonucleotides and Analogs, Ed. Agrawal (1993), Humana Press) and/or RNA like compounds (Scaringe, Methods (2001), 23, 206-217. Gait et al., Applications of Chemically synthesized RNA in RNA:Protein Interactions, Ed. Smith (1998), 1-36. Gallo et al., Tetrahedron (2001), 57, 5707-5713) synthesis as appropriate. In addition specific protocols for the synthesis of oligomeric compounds of the invention are illustrated in the examples below.

RNA oligomers can be synthesized by methods disclosed herein or purchased from various RNA synthesis companies such as for example Dharrnacon Research Inc., (Lafayette, Colo.).

Irrespective of the particular protocol used, the oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

For double stranded structures of the invention, once synthesized, the complementary strands preferably are annealed. The single strands are aliquoted and diluted to a concentration of 50 uM. Once diluted, 30 uL of each strand is combined with 15uL of a 5× solution of annealing buffer. The final concentration of the buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA compound is 20 uM. This solution can be stored frozen (−20° C.) and freeze-thawed up to 5 times.

Once prepared, the desired synthetic duplexes are evaluated for their ability to modulate target expression. When cells reach 80% confluency, they are treated with synthetic duplexes comprising at least one oligomeric compound of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired dsRNA compound at a final concentration of 200 nM. After 5 hours of treatment, the medium is replaced with fresh medium. Cells are harvested 16 hours after treatment, at which time RNA is isolated and target reduction measured by RT-PCR.

Oligomer and Monomer Modifications

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligomers, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligomers, the phosphate groups are commonly referred to as forming the internucleoside linkage or in conjunction with the sugar ring the backbone of the oligomer. The normal intemucleoside linkage that makes up the backbone of RNA and DNA is a 3′ to 5′ phosphodiester linkage.

Modified Internucleoside Linkages

Specific examples of preferred antisense oligomeric compounds useful in this invention include oligomers containing modified e.g. non-naturally occurring internucleoside linkages. As defined in this specification, oligomers having modified internucleoside linkages include internucleoside linkages that retain a phosphorus atom and internucleoside linkages that do not have a phosphorus atom. For the purposes of this specification, and as sometimes referenced in the art, modified oligomers that do not have a phosphorus atom in their internucleoside backbone can also be considered to be oligonucleosides.

In the C. elegans system, modification of the internucleotide linkage (phosphorothioate) did not significantly interfere with RNAi activity. Based on this observation, it is suggested that certain preferred oligomeric compounds of the invention can also have one or more modified internucleoside linkages. A preferred phosphorus containing modified internucleoside linkage is the phosphorothioate internucleoside linkage.

Preferred modified oligomer backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more intemucleotide linkages is a 3′ to 3′, 5′ to 5′ or 2′ to 2′ linkage. Preferred oligomers having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

In more preferred embodiments of the invention, oligomeric compounds have one or more phosphorothioate and/or heteroatom internucleoside linkages, in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester internucleotide linkage is represented as —O—P(═O)(OH)—O—CH₂—]. The MMI type internucleoside linkages are disclosed in the above referenced U.S. Pat. No. 5,489,677. Preferred amide internucleoside linkages are disclosed in the above referenced U.S. Pat. No. 5,602,240.

Preferred modified oligomer backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetal and thioformacetal backbones; methylene formacetal and thioformacetal backbones; riboacetal backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. No. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Additional Oligomer Mimetics

Another preferred group of oligomeric compounds amenable to the present invention includes oligonucleotide mimetics. The term mimetic as it is applied to oligomers is intended to include oligomeric compounds wherein only the furanose ring or both the furanose ring and the internucleotide linkage are replaced with novel groups, replacement of only the furanose ring is also referred to in the art as being a sugar surrogate. The heterocyclic base moiety or a modified heterocyclic base moiety is maintained for hybridization with an appropriate target nucleic acid.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566) and would have the general formula:

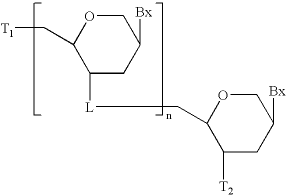

A further preferred modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage is preferably a methylene (—CH₂—)ₙ group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10 C), stability towards 3′-exonucleolytic degradation and good solubility properties. The basic structure of LNA showing the bicyclic ring system is shown below:

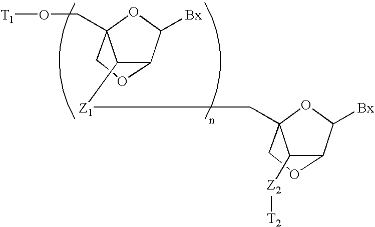

The conformations of LNAs determined by 2D NMR spectroscopy have shown that the locked orientation of the LNA nucleotides, both in single-stranded LNA and in duplexes, constrains the phosphate backbone in such a way as to introduce a higher population of the N-type conformation (Petersen et al., J. Mol. Recognit., 2000, 13, 44-53). These conformations are associated with improved stacking of the nucleobases (Wengel et al., Nucleosides Nucleotides, 1999, 18, 1365-1370).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands.

Novel types of LNA-oligomeric compounds, as well as the LNAs, are useful in a wide range of diagnostic and therapeutic applications. Among these are antisense applications, PCR applications, strand-displacement oligomers, substrates for nucleic acid polymerases and generally as nucleotide based drugs.

Potent and nontoxic antisense oligomers containing LNAs have been described (Wahlestedt et al., Proc. Natl. Acad. Sci. U.S.A., 2000, 97, 5633-5638.) The authors have demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

The first analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., PCT International Application WO 98-DK393 19980914). Furthermore, synthesis of 2′-amino-LNA, a novel conformationally restricted high-affinity oligonucleotide analog with a handle has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Further oligonucleotide mimetics have been prepared to include bicyclic and tricyclic nucleoside analogs having the formulas (amidite monomers shown): 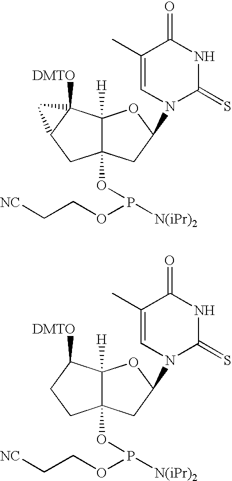
(see Steffens et al., Helv. Chim. Acta, 1997, 80, 2426-2439; Steffens et al., J. Am. Chem. Soc., 1999, 121, 3249-3255; and Renneberg et al., J. Am. Chem. Soc., 2002, 124, 5993-6002). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids incorporate a phosphorus group in a backbone the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

The general formula (for definitions of variables see: U.S. Pat. Nos. 5,874,553 and 6,127,346 herein incorporated by reference in their entirety) is shown below.

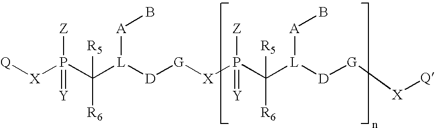

Another oligonucleotide mimetic has been reported wherein the furanosyl ring has been replaced by a cyclobutyl moiety.

Modified Sugars

Oligomeric compounds of the invention may also contain one or more substituted sugar moieties. Preferred oligomeric compounds comprise a sugar substituent group selected from: OH; F; O—, S—, or N-alkyl; O—, S—, or N— alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligomers comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligomer, or a group for improving the pharmacodynamic properties of an oligomer, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other preferred sugar substituent groups include methoxy (—O—CH₃), aminopropoxy (—OCH₂CH₂CH₂NH₂), allyl (—CH₂—CH═CH₂), —O-allyl (—O—CH₂—CH═CH₂) and fluoro (F). 2′-Sugar substituent groups may be in the arabino (up) position or ribo (down) position. A preferred 2′-arabino modification is 2′-F. Similar modifications may also be made at other positions on the oligomeric compound, particularly the 3′ position of the sugar on the 3′ terminal nucleoside or in 2′-5′ linked oligomers and the 5′ position of 5′ terminal nucleotide. Oligomeric compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Further representative sugar substituent groups include groups of formula I₁ a or IIₐ: 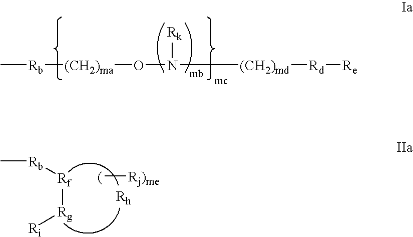
wherein:
R_{b} is O, S or NH;
R_{d} is a single bond, O, S or C(═O);
Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIₐ; 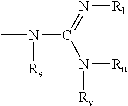
   IIₐ

Rₚ and R_{q} are each independently hydrogen or C₁-C₁₀ alkyl;
Rᵣ is —Rₓ—R_{y};
each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, Rᵤ and Rᵥ, together form a phthalimido moiety with the nitrogen atom to which they are attached;
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
Rₖ is hydrogen, a nitrogen protecting group or —Rₓ—R_{y};
Rₚ is hydrogen, a nitrogen protecting group or —Rₓ—R_{y};
Rₓ is a bond or a linking moiety;
R_{y} is a chemical functional group, a conjugate group or a solid support medium;
each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;
or Rₘ and Rₙ, together, are a nitrogen protecting group, are joined in a ring structure that optionally includes an additional heteroatom selected from N and O or are a chemical functional group;
Rᵢ is OR_{z}, SR_{z}, or N(R_{z})₂;
each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;
R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;
Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN;
mₐ is 1 to about 10;
each mb is, independently, 0 or 1;
mc is 0 or an integer from 1 to 10;
md is an integer from 1 to 10;
me is from 0, 1 or 2; and
provided that when mc is 0, md is greater than 1.

Representative substituents groups of Formula I are disclosed in U.S. patent application Ser. No. 09/130,973, filed Aug. 7, 1998, entitled “Capped 2′-Oxyethoxy Oligonucleotides,” hereby incorporated by reference in its entirety.

Representative cyclic substituent groups of Formula II are disclosed in U.S. patent application Ser. No. 09/123,108, filed Jul. 27, 1998, entitled “RNA Targeted 2′-Oligomeric compounds that are Conformationally Preorganized,” hereby incorporated by reference in its entirety.

Particularly preferred sugar substituent groups include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10.

Representative guanidino substituent groups that are shown in formula III and IV are disclosed in co-owned U.S. patent application Ser. No. 09/349,040, entitled “Functionalized Oligomers”, filed Jul. 7, 1999, hereby incorporated by reference in its entirety.

Representative acetamido substituent groups are disclosed in U.S. Pat. No. 6,147,200 which is hereby incorporated by reference in its entirety.

Representative dimethylaminoethyloxyethyl substituent groups are disclosed in International Patent Application PCT/US99/17895, entitled “2′-O-Dimethylaminoethyloxyethyl-Oligomeric compounds”, filed Aug. 6, 1999, hereby incorporated by reference in its entirety.

Modified Nucleobases/Naturally Occurring Nucleobases

Oligomeric compounds may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-0-methoxyethyl sugar modifications.

In one aspect of the present invention oligomeric compounds are prepared having polycyclic heterocyclic compounds in place of one or more heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Many of these polycyclic heterocyclic compounds have the general formula:

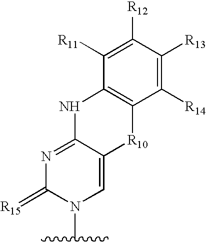

Representative cytosine analogs that make 3 hydrogen bonds with a guanosine in a second strand include 1,3-diazaphenoxazine-2-one (R₁₀₌O, R₁₁—R₁₄═H) [Kurchavov, et al., Nucleosides and Nucleotides, 1997, 16, 1837-1846], 1,3-diazaphenothiazine-2-one (R₁₀═S, R₁₁—R₁₄═H), [Lin, K.-Y.; Jones, R. J.; Matteucci, M. J. Am. Chem. Soc. 1995, 117, 3873-3874] and 6,7,8,9-tetrafluoro-1,3-diazaphenoxazine-2-one (R₁₀═O, R₁₁—R₁₄═F) [Wang, J.; Lin, K.-Y., Matteucci, M. Tetrahedron Lett. 1998, 39, 8385-8388]. Incorporated into oligonucleotides these base modifications were shown to hybridize with complementary guanine and the latter was also shown to hybridize with adenine and to enhance helical thermal stability by extended stacking interactions(also see U.S. patent application entitled “Modified Peptide Nucleic Acids” filed May 24, 2002, Ser. No. 10/155,920; and U.S. patent application entitled “Nuclease Resistant Chimeric Oligonucleotides” filed May 24, 2002, Ser. No. 10/013,295, both of which are commonly owned with this application and are herein incorporated by reference in their entirety).

Further helix-stabilizing properties have been observed when a cytosine analog/substitute has an aminoethoxy moiety attached to the rigid 1,3-diazaphenoxazine-2-one scaffold (R₁₀₌O, R₁₁═—O—(CH₂)₂—NH₂, R₁₂₋₁₄═H) [Lin, K.-Y.; Matteucci, M. J. Am. Chem. Soc. 1998, 120, 8531-8532]. Binding studies demonstrated that a single incorporation could enhance the binding affinity of a model oligonucleotide to its complementary target DNA or RNA with a ΔTm of up to 18° relative to 5-methyl cytosine (dC5^{me}), which is the highest known affinity enhancement for a single modification, yet. On the other hand, the gain in helical stability does not compromise the specificity of the oligomers. The Tdata indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in U.S. Pat. No. 6,028,183, which issued on May 22, 2000, and U.S. Pat. No. 6,007,992, which issued on Dec. 28, 1999, the contents of both are commonly assigned with this application and are incorporated herein in their entirety.

The enhanced binding affinity of the phenoxazine derivatives together with their uncompromised sequence specificity make them valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity [Lin, K-Y; Matteucci, M. J. Am. Chem. Soc. 1998, 120, 8531-8532]. The activity enhancement was even more pronounced in case of G-clamp, as a single substitution was shown to significantly improve the in vitro potency of a 20mer 2′-deoxyphosphorothioate oligonucleotides [Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518]. Nevertheless, to optimize oligomer design and to better understand the impact of these heterocyclic modifications on the biological activity, it is important to evaluate their effect on the nuclease stability of the oligomers.

Further modified polycyclic heterocyclic compounds useful as heterocyclcic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. patent application Ser. No. 09/996,292 filed Nov. 28, 2001, certain of which are commonly owned with the instant application, and each of which is herein incorporated by reference.

Conjugates

A further preferred substitution that can be appended to the oligomeric compounds of the invention involves the linkage of one or more moieties or conjugates which enhance the activity, cellular distribution or cellular uptake of the resulting oligomeric compounds. In one embodiment such modified oligomeric compounds are prepared by covalently attaching conjugate groups to functional groups such as hydroxyl or amino groups. Conjugate groups of the invention include intercalators, reporter molecules, polyamines, polyamides, poly-ethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

The oligomeric compounds of the invention may also be conjugated to active drug substances, for example, aspirin, warfarin, phenylbuta-zone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligomer conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

Chimeric Oligomeric Compounds

It is not necessary for all positions in an oligomeric compound to be uniformly modified, and in fact more than one of the aforementioned modifications may be incorporated in a single oligomeric compound or even at a single monomeric subunit such as a nucleoside within a oligomeric compound. The present invention also includes oligomeric compounds which are chimeric oligomeric compounds. “Chimeric” oligomeric compounds or “chimeras,” in the context of this invention, are oligomeric compounds that contain two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, oligonucleotide analogs, oligonucleosides and/or oligonucleotide mimetics as described above. Such oligomeric compounds have also been referred to in the art as hybrids hemimers, gapmers or inverted gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

3′-endo Modifications

In one aspect of the present invention oligomeric compounds include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA like nucleosides so that particular properties of an oligomeric compound can be enhanced while maintaining the desirable 3′-endo conformational geometry. There is an apparent preference for an RNA type duplex (A form helix, predominantly 3′-endo) as a requirement (e.g. trigger) of RNA interference which is supported in part by the fact that duplexes composed of 2′-deoxy-2′-F-nucleosides appears efficient in triggering RNAi response in the C. elegans system. Properties that are enhanced by using more stable 3′-endo nucleosides include but aren't limited to modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. The present invention provides oligomeric triggers of RNAi having one or more nucleosides modified in such a way as to favor a C₃′-endo type conformation.

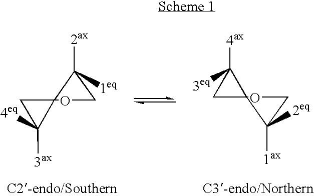

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′-endo conformation can be achieved while maintaining the 2′-OH as a recognition element, as illustrated in FIG. 2, below (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′ deoxy-2° F-nucleosides (Kawasaki et al., J. Med. Chem. (1993), 36, 831-841), which adopts the 3′-endo conformation positioning the electronegative fluorine atom in the axial position. Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Along similar lines, oligomeric triggers of RNAi response might be composed of one or more nucleosides modified in such a way that conformation is locked into a C₃′-endo type conformation, i.e. Locked Nucleic Acid (LNA, Singh et al, Chem. Commun. (1998), 4, 455456), and ethylene bridged Nucleic Acids (ENA, Morita et al, Bioorganic & Medicinal Chemistry Letters (2002), 12, 73-76.) Examples of modified nucleosides amenable to the present invention are shown below in Table I. These examples are meant to be representative and not exhaustive.

TABLE I

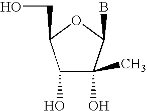

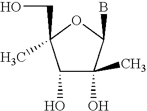

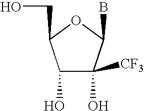

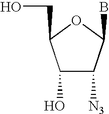

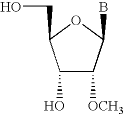

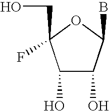

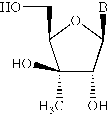

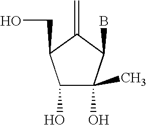

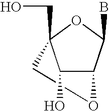

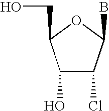

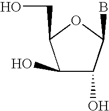

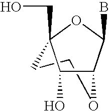

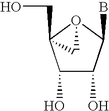

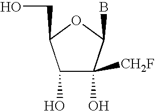

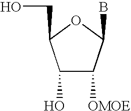

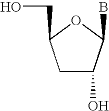

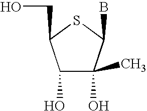

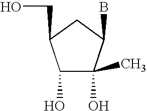

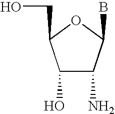

The preferred conformation of modified nucleosides and their oligomers can be estimated by various methods such as molecular dynamics calculations, nuclear magnetic resonance spectroscopy and CD measurements. Hence, modifications predicted to induce RNA like conformations, A-form duplex geometry in an oligomeric context, are selected for use in the modified oligomers of the present invention. The synthesis of numerous of the modified nucleosides amenable to the present invention are known in the art (see for example, Chemistry of Nucleosides and Nucleotides Vol 1-3, ed. Leroy B. Townsend, 1988, Plenum press., and the examples section below.) Nucleosides known to be inhibitors/substrates for RNA dependent RNA polymerases (for example HCV NS5B

In one aspect, the present invention is directed to oligomers that are prepared having enhanced properties compared to native RNA against nucleic acid targets. A target is identified and an oligomer is selected having an effective length and sequence that is complementary to a portion of the target sequence. Each nucleoside of the selected sequence is scrutinized for possible enhancing modifications. A preferred modification would be the replacement of one or more RNA nucleosides with nucleosides that have the same 3′-endo conformational geometry. Such modifications can enhance chemical and nuclease stability relative to native RNA while at the same time being much cheaper and easier to synthesize and/or incorporate into an oligomer. The selected sequence can be further divided into regions and the nucleosides of each region evaluated for enhancing modifications that can be the result of a chimeric configuration. Consideration is also given to the 5′ and 3′-termini as there are often advantageous modifications that can be made to one or more of the terminal nucleosides. The oligomeric compounds of the present invention include at least one 5′-modified phosphate group on a single strand or on at least one 5′-position of a double stranded sequence or sequences. Further modifications are also considered such as internucleoside linkages, conjugate groups, substitute sugars or bases, substitution of one or more nucleosides with nucleoside mimetics and any other modification that can enhance the selected sequence for its intended target.

The terms used to describe the conformational geometry of homoduplex nucleic acids are “A Form” for RNA and “B Form” for DNA. The respective conformational geometry for RNA and DNA duplexes was determined from X-ray diffraction analysis of nucleic acid fibers (Arnott and Hukins, Biochem. Biophys. Res. Comm., 1970,47, 1504.) In general, RNA:RNA duplexes are more stable and have higher melting temperatures (Tm's) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). As used herein, B-form geometry is inclusive of both C2′-endo pucker and O4′-endo pucker. This is consistent with Berger, et. al., Nucleic Acids Research, 1998, 26, 2473-2480, who pointed out that in considering the furanose conformations which give rise to B-form duplexes consideration should also be given to a O4′-endo pucker contribution.

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of the duplex formed between a target RNA and a synthetic sequence is central to therapies such as but not limited to antisense and RNA interference as these mechanisms require the binding of a synthetic oligomer strand to an RNA target strand. In the case of antisense, effective inhibition of the mRNA requires that the antisense DNA have a very high binding affinity with the mRNA. Otherwise the desired interaction between the synthetic oligomer strand and target mRNA strand will occur infrequently, resulting in decreased efficacy.

One routinely used method of modifying the sugar puckering is the substitution of the sugar at the 2′-position with a substituent group that influences the sugar geometry. The influence on ring conformation is dependant on the nature of the substituent at the 2′-position. A number of different substituents have been studied to determine their sugar puckering effect. For example, 2′-halogens have been studied showing that the 2′-fluoro derivative exhibits the largest population (65%) of the C₃′-endo form, and the 2′-iodo exhibits the lowest population (7%). The populations of adenosine (2′-OH) versus deoxyadenosine (2′-H) are 36% and 19%, respectively. Furthermore, the effect of the 2′-fluoro group of adenosine dimers (2′-deoxy-2′-fluoroadenosine-2′-deoxy-2′-fluoro-adenosine) is further correlated to the stabilization of the stacked conformation.

As expected, the relative duplex stability can be enhanced by replacement of 2′-OH groups with 2′-F groups thereby increasing the C₃′-endo population. It is assumed that the highly polar nature of the 2′-F bond and the extreme preference for C₃′-endo puckering may stabilize the stacked conformation in an A-form duplex. Data from UV hypochromicity, circular dichroism, and ¹H NMR also indicate that the degree of stacking decreases as the electronegativity of the halo substituent decreases. Furthermore, steric bulk at the 2′-position of the sugar moiety is better accommodated in an A-form duplex than a B-form duplex. Thus, a 2′-substituent on the 3'terminus of a dinucleoside monophosphate is thought to exert a number of effects on the stacking conformation: steric repulsion, furanose puckering preference, electrostatic repulsion, hydrophobic attraction, and hydrogen bonding capabilities. These substituent effects are thought to be determined by the molecular size, electronegativity, and hydrophobicity of the substituent. Melting temperatures of complementary strands is also increased with the 2′-substituted adenosine diphosphates. It is not clear whether the 3′-endo preference of the conformation or the presence of the substituent is responsible for the increased binding. However, greater overlap of adjacent bases (stacking) can be achieved with the 3′-endo conformation.

One synthetic 2′-modification that imparts increased nuclease resistance and a very high binding affinity to nucleotides is the 2-methoxyethoxy (2′-MOE, 2′-OCH₂CH₂OCH₃) side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-O-methoxyethyl substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926). Relative to DNA, the oligomers having the 2′-MOE modification displayed improved RNA affinity and higher nuclease resistance. Chimeric oligomers having 2′-MOE substituents in the wing nucleosides and an internal region of deoxy-phosphorothioate nucleotides (also termed a gapped oligomer or gapmer) have shown effective reduction in the growth of tumors in animal models at low doses. 2′-MOE substituted oligomers have also shown outstanding promise as antisense agents in several disease states. One such MOE substituted oligomer is presently being investigated in clinical trials for the treatment of CMV retinitis.

Chemistries Defined

Unless otherwise defined herein, alkyl means C₁-C₁₂, preferably C₁-C₈, and more preferably C₁-C₆, straight or (where possible) branched chain aliphatic hydrocarbyl.

Unless otherwise defined herein, heteroalkyl means C₁-C₁₂, preferably C₁-C₈, and more preferably C₁-C₆, straight or (where possible) branched chain aliphatic hydrocarbyl containing at least one, and preferably about 1 to about 3, hetero atoms in the chain, including the terminal portion of the chain. Preferred heteroatoms include N, O and S.

Unless otherwise defined herein, cycloalkyl means C₃-C₁₂, preferably C₃-C₈, and more preferably C₃-C₆, aliphatic hydrocarbyl ring.

Unless otherwise defined herein, alkenyl means C₂-C₁₂, preferably C₂-C₈, and more preferably C₂-C₆ alkenyl, which may be straight or (where possible) branched hydrocarbyl moiety, which contains at least one carbon-carbon double bond.

Unless otherwise defined herein, alkynyl means C₂-C₁₂, preferably C₂-C₈, and more preferably C₂-C₆ alkynyl, which may be straight or (where possible) branched hydrocarbyl moiety, which contains at least one carbon-carbon triple bond.

Unless otherwise defined herein, heterocycloalkyl means a ring moiety containing at least three ring members, at least one of which is carbon, and of which 1, 2 or three ring members are other than carbon. Preferably the number of carbon atoms varies from 1 to about 12, preferably 1 to about 6, and the total number of ring members varies from three to about 15, preferably from about 3 to about 8. Preferred ring heteroatoms are N, O and S. Preferred heterocycloalkyl groups include morpholino, thiomorpholino, piperidinyl, piperazinyl, homopiperidinyl, homopiperazinyl, homomorpholino, homothiomorpholino, pyrrolodinyl, tetrahydrooxazolyl, tetrahydroimidazolyl, tetrahydrothiazolyl, tetrahydroisoxazolyl, tetrahydropyrrazolyl, furanyl, pyranyl, and tetrahydroisothiazolyl.

Unless otherwise defined herein, aryl means any hydrocarbon ring structure containing at least one aryl ring. Preferred aryl rings have about 6 to about 20 ring carbons. Especially preferred aryl rings include phenyl, napthyl, anthracenyl, and phenanthrenyl.

Unless otherwise defined herein, hetaryl means a ring moiety containing at least one fully unsaturated ring, the ring consisting of carbon and non-carbon atoms. Preferably the ring system contains about 1 to about 4 rings. Preferably the number of carbon atoms varies from 1 to about 12, preferably 1 to about 6, and the total number of ring members varies from three to about 15, preferably from about 3 to about 8. Preferred ring heteroatoms are N, O and S. Preferred hetaryl moieties include pyrazolyl, thiophenyl, pyridyl, imidazolyl, tetrazolyl, pyridyl, pyrimidinyl, purinyl, quinazolinyl, quinoxalinyl, benzimidazolyl, benzothiophenyl, etc.

Unless otherwise defined herein, where a moiety is defined as a compound moiety, such as hetarylalkyl (hetaryl and alkyl), aralkyl (aryl and alkyl), etc., each of the sub-moieties is as defined herein.

Unless otherwise defined herein, an electron withdrawing group is a group, such as the cyano or isocyanato group that draws electronic charge away from the carbon to which it is attached. Other electron withdrawing groups of note include those whose electronegativities exceed that of carbon, for example halogen, nitro, or phenyl substituted in the ortho- or para-position with one or more cyano, isothiocyanato, nitro or halo groups.

Unless otherwise defined herein, the terms halogen and halo have their ordinary meanings. Preferred halo (halogen) substituents are Cl, Br, and I. The aforementioned optional substituents are, unless otherwise herein defined, suitable substituents depending upon desired properties. Included are halogens (Cl, Br, I), alkyl, alkenyl, and alkynyl moieties, NO₂, NH₃ (substituted and unsubstituted), acid moieties (e.g. —CO₂H, —OSO₃H₂, etc.), heterocycloalkyl moieties, hetaryl moieties, aryl moieties, etc. In all the preceding formulae, the squiggle (˜) indicates a bond to an oxygen or sulfur of the 5′-phosphate.

Phosphate protecting groups include those described in U.S. Pat. Nos. 5,760,209, 5,614,621, 6,051,699, 6,020,475, 6,326,478, 6,169,177, 6,121,437, 6,465,628 each of which is expressly incorporated herein by reference in its entirety.

Phosphotioate groups include those described in U.S. Pat. Nos. 3,687,808, 5,188,897, 5,278,302, 5,286,717, 5,405,939, 5,453,496, and 5,587,361.

Alkylphosphoroamidate groups include those described in U.S. Pat. No. 5,536,821 and 5,541,306,

Unless otherwise defined herein, alkoxy is defined as —O-alkyl where alkyl is as defined above.

Unless otherwise defined herein, alkylthio is defined as —S-alkyl where alkyl is as defined above.

Screening, Target Validation and Drug Discovery

For use in screening and target validation, the compounds and compositions of the invention are used to modulate the expression of a selected protein. “Modulators” are those oligomeric compounds and compositions that decrease or increase the expression of a nucleic acid molecule encoding a protein and which comprise at least an 8-nucleobase portion which is complementary to a preferred target segment. The screening method comprises the steps of contacting a preferred target segment of a nucleic acid molecule encoding a protein with one or more candidate modulators, and selecting for one or more candidate modulators which decrease or increase the expression of a nucleic acid molecule encoding a protein. Once it is shown that the candidate modulator or modulators are capable of modulating (e.g. either decreasing or increasing) the expression of a nucleic acid molecule encoding a peptide, the modulator may then be employed in further investigative studies of the function of the peptide, or for use as a research, diagnostic, or therapeutic agent in accordance with the present invention.

The conduction such screening and target validation studies, oligomeric compounds of invention can be used combined with their respective complementary strand oligomeric compound to form stabilized double-stranded (duplexed) oligomers. Double stranded oligomer moieties have been shown to modulate target expression and regulate translation as well as RNA processing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200; Nishikura et al., Cell (2001), 107, 415-416; and Bass et al., Cell (2000), 101, 235-238.) For example, such double-stranded moieties have been shown to inhibit the target by the classical hybridization of antisense strand of the duplex to the target, thereby triggering enzymatic degradation of the target (Tijsterman et al., Science, 2002, 295, 694-697).

For use in drug discovery and target validation, oligomeric compounds of the present invention are used to elucidate relationships that exist between proteins and a disease state, phenotype, or condition. These methods include detecting or modulating a target peptide comprising contacting a sample, tissue, cell, or organism with the oligomeric compounds and compositions of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a disease or disorder.

Kits, Research Reagents, Diagnostics, and Therapeutics

The oligomeric compounds and compositions of the present invention can additionally be utilized for diagnostics, therapeutics, prophylaxis and as research reagents and kits. Such uses allows for those of ordinary skill to elucidate the function of particular genes or to distinguish between functions of various members of a biological pathway.

For use in kits and diagnostics, the oligomeric compounds and compositions of the present invention, either alone or in combination with other compounds or therapeutics, can be used as tools in differential and/or combinatorial analyses to elucidate expression patterns of a portion or the entire complement of genes expressed within cells and tissues.

As one non-limiting example, expression patterns within cells or tissues treated with one or more compounds or compositions of the invention are compared to control cells or tissues not treated with the compounds or compositions and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds that affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000,480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High 7hroughput Screen, 2000, 3, 235-41).

The compounds and compositions of the invention are useful for research and diagnostics, because these compounds and compositions hybridize to nucleic acids encoding proteins. Hybridization of the compounds and compositions of the invention with a nucleic acid can be detected by means known in the art. Such means may include conjugation of an enzyme to the compound or composition, radiolabelling or any other suitable detection means. Kits using such detection means for detecting the level of selected proteins in a sample may also be prepared.

The specificity and sensitivity of compounds and compositions can also be harnessed by those of skill in the art for therapeutic uses. Antisense oligomeric compounds have been employed as therapeutic moieties in the treatment of disease states in animals, including humans. Antisense oligomer drugs, including ribozymes, have been safely and effectively administered to humans and numerous clinical trials are presently underway. It is thus established that oligomeric compounds can be useful therapeutic modalities that can be configured to be useful in treatment regimes for the treatment of cells, tissues and animals, especially humans.

For therapeutics, an animal, preferably a human, suspected of having a disease or disorder that can be treated by modulating the expression of a selected protein is treated by administering the compounds and compositions. For example, in one non-limiting embodiment, the methods comprise the step of administering to the animal in need of treatment, a therapeutically effective amount of a protein inhibitor. The protein inhibitors of the present invention effectively inhibit the activity of the protein or inhibit the expression of the protein. In one embodiment, the activity or expression of a protein in an animal is inhibited by about 10%. Preferably, the activity or expression of a protein in an animal is inhibited by about 30%. More preferably, the activity or expression of a protein in an animal is inhibited by 50% or more.

For example, the reduction of the expression of a protein may be measured in serum, adipose tissue, liver or any other body fluid, tissue or organ of the animal. Preferably, the cells contained within the fluids, tissues or organs being analyzed contain a nucleic acid molecule encoding a protein and/or the protein itself.

The compounds and compositions of the invention can be utilized in pharmaceutical compositions by adding an effective amount of the compound or composition to a suitable pharmaceutically acceptable diluent or carrier. Use of the oligomeric compounds and methods of the invention may also be useful prophylactically.

Formulations

The compounds and compositions of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The compounds and compositions of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the oligomeric compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligomers of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds and compositions of the invention: i.e., salts that retain the desired biological activity of the parent compound and do not impart undesired toxicological effects thereto. For oligomers, preferred examples of pharmaceutically acceptable salts and their uses are further described in U.S. Pat. No. 6,287,860, which is incorporated herein in its entirety.

The present invention also includes pharmaceutical compositions and formulations that include the compounds and compositions of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compounds and compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug that may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the present invention may also include surfactants. The use of surfactants in drug products, formulations and in emulsions is well known in the art. Surfactants and their uses are further described in U.S. Pat. No. 6,287,860, which is incorporated herein in its entirety.

In one embodiment, the present invention employs various penetration enhancers to effect the efficient delivery of nucleic acids, particularly oligomers. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligomers of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, compounds and compositions of the invention may be encapsulated within liposomes or may form complexes thereto, in particular to cationic liposomes. Alternatively, they may be complexed to lipids, in particular to cationic lipids. Preferred fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligomers of the invention are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Compounds and compositions of the invention may be delivered orally, in granular form including sprayed dried particles, or complexed to form micro or nanoparticles. Complexing agents and their uses are further described in U.S. Pat. No. 6,287,860, which is incorporated herein in its entirety. Certain oral formulations for oligomers and their preparation are described in detail in U.S. applications Ser. No. 09/108,673 (filed Jul. 1, 1998), Ser. No. 09/315,298 (filed May 20, 1999) and Ser. No. 10/071,822, filed Feb. 8, 2002, each of which is incorporated herein by reference in their entirety.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more of the compounds and compositions of the invention and one or more other chemotherapeutic agents that function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the oligomeric compounds of the invention, such chemo-therapeutic agents may be used individually (e.g., 5-FU and oligomer), sequentially (e.g., 5-FU and oligomer for a period of time followed by MTX and oligomer), or in combination with one or more other such chemotherapeutic agents (e.g., 5-FU, MTX and oligomer, or 5-FU, radiotherapy and oligomer). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of compounds and compositions of the invention and other drugs are also within the scope of this invention. Two or more combined compounds such as two oligomeric compounds or one oligomeric compound combined with further compounds may be used together or sequentially.

In another related embodiment, compositions of the invention may contain one or more of the compounds and compositions of the invention targeted to a first nucleic acid and one or more additional compounds such as antisense oligomeric compounds targeted to a second nucleic acid target. Numerous examples of antisense oligomeric compounds are known in the art. Alternatively, compositions of the invention may contain two or more oligomeric compounds and compositions targeted to different regions of the same nucleic acid target. Two or more combined compounds may be used together or sequentially

Dosing

The formulation of therapeutic compounds and compositions of the invention and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligomers, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligomer is administered in maintenance doses, ranging from 0.01 ug to 100 g per kg of body weight, once or more daily, to once every 20 years.

While the present invention has been described with specificity in accordance with certain of its preferred embodiments, the following examples serve only to illustrate the invention and are not intended to limit the same.

The entire disclosure of each patent, patent application, and publication cited or described in this document is hereby incorporated by reference.

### EXAMPLE 1

### Synthesis of Nucleoside Phosphoramidites

The following compounds, including amidites and their intermediates were prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N4-benzoyl-5-methylcytidine penultimate intermediate for 5-methyl dC amidite, [5′-O-(4,4′-Dimethoxytriphenylmethyl)-2′-deoxy-N⁴-benzoyl-5-methylcytidin-3 ′-O-yl]-2-cyanoethyl-N,N-diisopropylphosphoramidite(5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, [5′-O-(4,4′-Dimethoxytriphenylmethyl)-2′-O-(2-methoxyethyl)-5-methyluridin-3′-O-yl]-2-cyanoethyl-N,N-diisopropylphosphoramidite(MOE T amidite), 5′-O-Dimethoxytrityl-2′-O-(2-methoxyethyl)-5-methylcytidine intermediate, 5′-O-dimethoxytrityl-2′-O-(2-methoxyethyl)-N⁴-benzoyl-5-methyl-cytidine penultimate intermediate, [5′-O-(4,4′-Dimethoxytriphenylmethyl)-2′-O-(2-methoxyethyl)-N⁴-benzoyl-5-methylcytidin-3 ′-O-yl]-2-cyanoethyl-N,N-diisopropylphosphoramidite(MOE 5-Me-C amidite), [5′-O-(4,4′-Dimethoxytriphenylmethyl)-2′-O-(2-methoxyethyl)-N⁶-benzoyladenosin-3 ′-O-yl]-2-cyanoethyl-N,N-diisopropylphosphoramidite(MOE A amdite), [5′-O-(4,4′-Dimethoxytriphenylmethyl)-2′-O-(2-methoxyethyl)-N⁴-isobutyrylguanosin-3′-O-yl]-2-cyanoethyl-N,N-diisopropylphosphoramidite(MOE G amidite), 2′-O-(Aminooxyethyl)nucleoside amidites and 2′-O-(dimethylaminooxyethyl) nucleoside amidites, 2′-(Dimethylaminooxyethoxy) nucleoside amidites, 5′-O-tert-Butyldiphenylsilyl-O²-2′-anhydro-5-methyluridine, 5′-O-tert-Butyldiphenylsilyl-2′-O-(2-hydroxyethyl)-5-methyluridine, 2′-O-([2-phthalimidoxy)ethyl]-5′-t-butyldiphenylsilyl-5-methyluridine, 5′-O-tert-butyldiphenylsilyl-2′-O-[(2-formadoximinooxy)ethyl]-5-methyluridine, 5′-O-tert-Butyldiphenylsilyl-2′-O-[N,N dimethylaminooxyethyl]-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-[(2-cyanoethyl)-N,N-diisopropylphosphoramidite], 2′-(Aminooxyethoxy)nucleoside amidites, N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite], 2′-dimethylaminoethoxyethoxy(2′-DMAEOE)nucleoside amidites, 2′-O-[2(2-N,N-dimethylaminoethoxy)ethyl]-5-methyl uridine, 5′-O-dimethoxytrityl-2′-O-[2(2-N,N-dimethylaminoethoxy)-ethyl)]-5-methyl uridine and 5′-O-Dimethoxytrityl-2′-O-[2(2-N,N-dimethylaminoethoxy)-ethyl)]-5-methyl uridine-3′-O-(cyanoethyl-N,N-diisopropyl)phosphoramidite.

### EXAMPLE 2

### Oligomer, Oligonucleotide and Oligonucleoside Synthesis

Oligomers such as oligonucleotides: Unsubstituted and substituted phosphodiester (P═O) oligomers are synthesized on an automated DNA synthesizer (Applied Biosystems model 394) using standard phosphoramidite chemistry with oxidation by iodine.

Phosphorothioates (P═S) are synthesized similar to phosphodiester oligomers with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligomers were recovered by precipitating with >3 volumes of ethanol from a 1 M NH₄OAc solution. Phosphinate oligomers are prepared as described in U.S. Pat. No. 5,508,270, herein incorporated by reference.

Alkyl phosphonate oligomers are prepared as described in U.S. Pat. No. 4,469,863, herein incorporated by reference.

3′-Deoxy-3′-methylene phosphonate oligomers are prepared as described in U.S. Pat. Nos. 5,610,289 or 5,625,050, herein incorporated by reference.

Phosphorarnidite oligomers are prepared as described in U.S. Pat. No. 5,256,775 or U.S. Pat. No. 5,366,878, herein incorporated by reference.

Alkylphosphonothioate oligomers are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligomers are prepared as described in U.S. Pat. No. 5,476,925, herein incorporated by reference.

Phosphotriester oligomers are prepared as described in U.S. Pat. No. 5,023,243, herein incorporated by reference.

Borano phosphate oligomers are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedi-methylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone oligomeric compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### EXAMPLE 3

### RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligomers were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligomer synthesis. However, after oligomer synthesis the oligomer is treated with methylamine which not only cleaves the oligomer from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligomer synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

### EXAMPLE 4

### Synthesis of Chimeric Oligomers

Chimeric oligomers, oligonucleotides, oligonucleosides or mixed oligonucleotides/oligonucleosides of the invention can be of several different types. These include a first type wherein the “gap” segment of linked nucleosides is positioned between 5′ and 3“wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

### [2′-O-Me]-[2′-deoxy]-[2′-O-Me]Chimeric Phosphorothioate Oligomers

Chimeric oligomers having 2′-O-alkyl phosphorothioate and 2′-deoxy phosphorothioate oligomer segments are synthesized using an Applied Biosystems automated DNA synthesizer Model 394, as above. Oligomers are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxy-trityl-2′-O-methyl-3′-O-phosphoramidite. The fully protected oligomer is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

### [2′-O-(2-Methoxyethyl))-[2′-deoxy]-[2′-O-(Methoxyethyl)]Chimeric

Phosphorothioate Oligomers

[2′-O-(2-methoxyethyl)]-[2′-deoxy]-[-2′-O-(methoxyethyl)]chimeric phosphorothioate oligomers were prepared as per the procedure above for the 2′-O-methyl chimeric oligomer, with the substitution of 2′-O-(methoxyethyl)amidites for the 2′-O-methyl amidites.

### [2′-O-(2-Methoxyethyl)Phosphodiester]-[2′-deoxy Phosphorothioate]-[2′-O-(2-Methoxyethyl) Phosphodiester]Chimeric Oligomers

[2′-O-(2-methoxyethyl phosphodiester]-[2′-deoxy phosphorothioate]-[2′-O-(methoxyethyl)phosphodiester]chimeric oligomers are prepared as per the above procedure for the 2′-O-methyl chimeric oligomer with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligomers, chimeric oligonucleosides and mixed chimeric oligomers/oligonucleosides are synthesized according to U.S. Pat. No. 5,623,065, herein incorporated by reference.

### EXMAPLE 5

### General Synthesis of Morpholino Nucleic Acids

Morpholino nucleic acids are synthesized by methods taught in U.S. Pat. Nos. 5,405,938, 5,235,033, 5,188,879, and 5,166,315.

### EXAMPLE 6

### Synthesis of Alpha Morpholino Ribonucleosides with Cleavable R Group

These compounds are synthesized by the methods of U.S. Pat. No. 5,235,033.

### EXAMPLE 7

### Synthesis of N-Carboxymethylmorpholino-5′-amino Backbone Oligomers

These compounds are synthesized by the methods of U.S. Pat. No. 5,166,315.

### EXAMPLE 8

### General Synthesis of Peptide Nucleic Acids

Peptide nucleic acids are synthesized by methods taught in U.S. Pat. Nos. 5,539,082, 5,714,331, 5,719,262 and 6,395,474.

### EXAMPLE 9

### Synthesis of N′-Boc-Aminoethylglycine Methyl Ester

This compound is synthesized by the method of U.S. Pat. No. 6,395,474.

### EXAMPLE 10

### Synthesis of N-[(1-Thyminyl)acetyl]-N′-Boc-Amino-β-Alanine

This compound is synthesized by the method of U.S. Pat. No. 6,395,474.

### EXAMPLE 11

### Synthesis of Diethyl N-thymin-1-yl-acetyl-N-(4-methoxytriphenylmethoxy)ethylaminomethanephosphonate

This compound is synthesized by the method taught in U.S. Pat. No. 6,127,346.

### EXAMPLE 12

### Synthesis of N-thymin-1-yl-acetyl-N-(4-methoxytriphenylmethoxy)ethylaminomethanephosphonic acid monoethyl ester(triethylammonium salt)

This compound is synthesized by the method taught in U.S. Pat. No. 6,127,346.

### EXAMPLE 13

### Synthesis of H-[Aeg(A)]-[Aeg(C)]-[Aeg(A)]-[Aeg(T)]-[Aeg(C)]-[Aeg(A)]-[Aeg(T)]-[Aeg(G)]-[Aeg(G)]-[Aeg(T)]-[Aeg(C)]-[Aeg(G) (hex)

This PNA is synthesized by the method of U.S. Pat. No. 6,046,306.

### EXAMPLE 14

### Synthesis of Oligonucleotides Containing Internucleoside Linkages Comprised of a Hexose Sugar and an Amide

Oligonucleotides containing internucleoside linkages comprised of a hexose sugar and an amide are synthesized as described in U.S. Pat. No. 5,780,607.

### EXAMPLE 15

### Synethsis of Cyclohexene Nucleic Acids

Cyclohexene nucleic acids are synthesized by the method of Wang et al., J. Am. Chem. Soc. 2000, 122, 8595-8602.

### EXAMPLE 16

### Synethesis of a Carbonate-Linked Acyclic Backbone Moiety

Carbonate-linked acyclic backbone moieties can be synthesized by the methods taught by PCT Patent Application No. 86/05518.

### EXAMPLE 17

### Design and Screening of Duplexed Oligomeric Compounds Targeting a Target

In accordance with the present invention, a series of nucleic acid duplexes comprising the antisense oligomeric compounds of the present invention and their complements can be designed to target a target. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG (SEQ ID NO:1) and having a two-nucleobase overhang of deoxythymidine(dT) would have the following structure:

| | | |
|---|---|---|
| 5′ c g a g a g g c g g a c g g g a c c g T T 3′ | Antisense Strand | (SEQ ID NO:2) |
| \| \|\| \| \| \| \| \| \| \|\| \| \| \| \| \| \| \| \| | | |
| 3′ T T g c t c t c c g c c t g c c c t g g c 5′ | Complément Strand | (SEQ ID NO:3) |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense oligomeric compounds are evaluated for their ability to modulate a target expression.

When cells reached 80% confluency, they are treated with duplexed antisense oligomeric compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex antisense oligomeric compound at a final concentration of 200 nM. After 5 hours of treatment, the medium is replaced with fresh medium. Cells are harvested 16 hours after treatment, at which time RNA is isolated and target reduction measured by RT-PCR.

### EXAMPLE 18

### Oligomer Isolation

After cleavage from the controlled pore glass solid support and deblocking in concentrated ammonium hydroxide at 55° C. for 12-16 hours, the oligomers or oligonucleosides are recovered by precipitation out of 1 M NH₄OAc with >3 volumes of ethanol. Synthesized oligomers were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the—16 amu product (±32±48). For some studies oligomers were purified by HPLC, as described by Chiang et al., J. Biol. Chem. 1991, 266, 18162-18171. Results obtained with HPLC-purified material were similar to those obtained with non-HPLC purified material.

### EXAMPLE 19

### Oligomer Synthesis—96 Well Plate Format

Oligomers were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligomers were cleaved from support and deprotected with concentrated NH₄OH at elevated temperature (55-60° C. ) for 12-16 hours and the released product then dried in vacuo. The dried product was then re-suspended in sterile water to afford a master plate from which all analytical and test plate samples are then diluted utilizing robotic pipettors.

### EXAMPLE 20

### Oligomer Analysis—96-Well Plate Format

The concentration of oligomer in each well was assessed by dilution of samples and UV absorption spectroscopy. The full-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the oligomeric compounds utilizing electrospray-mass spectroscopy. All assay test plates were diluted from the master plate using single and multi-channel robotic pipettors. Plates were judged to be acceptable if at least 85% of the oligomeric compounds on the plate were at least 85% full length.

### EXAMPLE 21

### Cell Culture and Oligomer Treatment

The effect of oligomeric compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells may be seeded onto 100 mm or other standard tissue culture plates and treated similarly, using appropriate volumes of medium and oligomer.

A549 cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF cells:

Human neonatal dermal fibroblast (NHDF) were obtained from the Clonetics Corporation (Walkersville, MD). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK cells:

Human embryonic keratinocytes (HEK) were obtained from the Clonetics Corporation (Walkersville, Md. HEKs were routinely maintained in Keratinocyte Growth Medium (Clonetics Corporation, Walkersville, Md.) formulated as recommended by the supplier. Cells were routinely maintained for up to 10 passages as recommended by the supplier.

Treatment with Antisense Oligomeric Compounds:

When cells reached 65-75% confluency, they were treated with oligomer. For cells grown in 96-well plates, wells were washed once with 100 μL OPTI-MEM™-1 reduced-serum medium (Invitrogen Corporation, Carlsbad, Calif.) and then treated with 130 μL of OPTI-ME™-1 containing 3.75 μg/mL LIPOFECTIN™ (Invitrogen Corporation, Carlsbad, Calif.) and the desired concentration of oligomer. Cells are treated and data are obtained in triplicate. After 4-7 hours of treatment at 37° C., the medium was replaced with fresh medium. Cells were harvested 16-24 hours after oligomer treatment.

The concentration of oligomer used varies from cell line to cell line. To determine the optimal oligomer concentration for a particular cell line, the cells are treated with a positive control oligomer at a range of concentrations. For human cells the positive control oligomer is selected from either ISIS 13920 (TCCGTCATCGCTCCTCAGGG, SEQ ID NO: 4) which is targeted to human H-ras, or ISIS 18078, (GTGCGCGCGAGCCCGAAATC, SEQ ID NO: 5) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligomer is ISIS 15770, ATGCATTCTGCCCCCAAGGA (SEQ ID NO: 6) a 2′-O-methoxyethyl gapmer (2′-O-methoxyethyls shown in bold) with a phosphorothioate backbone which is targeted to both mouse and rat c-raf. The concentration of positive control oligomer that results in 80% inhibition of c-H-ras (for ISIS 13920), JNK2 (for ISIS 18078) or c-raf (for ISIS 15770) mRNA is then utilized as the screening concentration for new oligomers in subsequent experiments for that cell line. If 80% inhibition is not achieved, the lowest concentration of positive control oligomer that results in 60% inhibition of c-H-ras, JNK2 or c-raf mRNA is then utilized as the oligomer screening concentration in subsequent experiments for that cell line. If 60% inhibition is not achieved, that particular cell line is deemed as unsuitable for oligomer transfection experiments. The concentrations of antisense oligomers used herein are from 50 nM to 300 nM.

### EXAMPLE 22

### Analysis of Oligomer Inhibition of a Target Expression

Modulation of a target expression can be assayed in a variety of ways known in the art. For example, a target mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. The preferred method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRIS™ 7600, 7700, or 7900 Sequence Detection System, available from PE-Applied Biosystems, Foster City, Calif. and used according to manufacturer's instructions.

Protein levels of a target can be quantitated in a variety of ways well known in the art, such as immunoprecipitation, Western blot analysis (immunoblotting), enzyme-linked immunosorbent assay (ELISA) or fluorescence-activated 0cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### EXAMPLE 23

### Design of Phenotypic Assays and In Vivo Studies for the Use of a Target Inhibitors

Phenotypic Assays

Once a target inhibitors have been identified by the methods disclosed herein, the oligomeric compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of a target in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-lirniting example, cells determined to be appropriate for a particular phenotypic assay (i.e., MCF-7 cells selected for breast cancer studies; adipocytes for obesity studies) are treated with a target inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints. Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the geneotype of the cell (measurement of the expression of one or more of the genes of the cell) after treatment is also used as an indicator of the efficacy or potency of the target inhibitors. Hallmark genes, or those genes suspected to be associated with a specific disease state, condition, or phenotype, are measured in both treated and untreated cells.

In Vivo Studies

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study.

To account for the psychological effects of receiving treatments, volunteers are randomly given placebo or a target inhibitor. Furthermore, to prevent the doctors from being biased in treatments, they are not informed as to whether the medication they are administering is a a target inhibitor or a placebo. Using this randomization approach, each volunteer has the same chance of being given either the new treatment or the placebo.

Volunteers receive either the a target inhibitor or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding a target or a target protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and a target inhibitor treatment. In general, the volunteers treated with placebo have little or no response to treatment, whereas the volunteers treated with the target inhibitor show positive trends in their disease state or condition index at the conclusion of the study.

### EXAMPLE 24

### RNA Isolation

Poly(A)+ mRNA Isolation

Poly(A)+ mRNA was isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine CA). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### EXAMPLE 25

### Real-Time Quantitative PCR Analysis of a Target mRNA Levels

Quantitation of a target mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligomer probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligomer treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5× PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5× ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

Probes and primers are designed to hybridize to a human a target sequence, using published sequence information.

### EXAMPLE 26

### Northern Blot Analysis of a Target mRNA Levels

Eighteen hours after treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human a target, a human a target specific primer probe set is prepared by PCR To normalize for variations in loading and transfer efficiency membranes are stripped and probed for human glyceraldehyde-3-phosphate dehydrogenase (GAPDH) RNA (Clontech, Palo Alto, Calif.).

Hybridized membranes were visualized and quantitated using a PHOSPHORMAGER™ and IMAGEQUAN™ Software V3.3 (Molecular Dynamics, Sunnyvale, Calif.). Data was normalized to GAPDH levels in untreated controls.

### EXAMPLE 27

### Inhibition of Human a Target Expression by Oligomers

In accordance with the present invention, a series of oligomeric compounds are designed to target different regions of the human target RNA. The oligomeric compounds are analyzed for their effect on human target mRNA levels by quantitative real-time PCR as described in other examples herein. Data are averages from three experiments. The target regions to which these preferred sequences are complementary are herein referred to as “preferred target segments” and are therefore preferred for targeting by oligomeric compounds of the present invention. The sequences represent the reverse complement of the preferred antisense oligomeric compounds.

As these “preferred target segments” have been found by experimentation to be open to, and accessible for, hybridization with the antisense oligomeric compounds of the present invention, one of skill in the art will recognize or be able to ascertain, using no more than routine experimentation, further embodiments of the invention that encompass other oligomeric compounds that specifically hybridize to these preferred target segments and consequently inhibit the expression of a target.

According to the present invention, antisense oligomeric compounds include antisense oligomeric compounds, antisense oligomers, ribozymes, external guide sequence (EGS) oligomers, alternate splicers, primers, probes, and other short oligomeric compounds that hybridize to at least a portion of the target nucleic acid.

### EXAMPLE 28

### Western Blot Analysis of a Target Protein Levels

Western blot analysis (immunoblot analysis) is carried out using standard methods. Cells are harvested 16-20 h after oligomer treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to a target is used, with a radiolabeled or fluorescently labeled secondary antibody directed against the primary antibody species. Bands are visualized using a PHOSPHORIMAGER™ (Molecular Dynamics, Sunnyvale Calif.).

1. A composition comprising a first oligomer and a second oligomer, wherein:
at least a portion of said first oligomer is capable of hybridizing with at least a portion of said second oligomer,
at least a portion of first oligomer is complementary to and capable of hybridizing to a selected target nucleic acid,
at least one of said first or said second oligomers includes at least one nucleotide having a modification comprising a peptide nucleic acid, a peptide nucleic acid mimic, a morpholino nucleic acid, hexose sugar with amide linkage, cyclohexenyl nucleic acid (CeNA) or an acyclic backbone moiety.

2. The composition of claim 1 wherein said first and said second oligomers are a complementary pair of siRNA oligomers.

3. The composition of claim 1 wherein said first and said second oligomers are an antisense/sense pair of oligomers.

4. The composition of claim 1 wherein each of said first and second oligomers has 10 to 40 nucleotides.

5. The composition of claim 1 wherein each of said first and second oligomers has 18 to 30 nucleotides.

6. The oligomer composition of claim 1 wherein each of said first and second oligomers has 21 to 24 nucleotides.

7. The composition of claim 1 wherein said first oligomer is an antisense oligomer.

8. The composition of claim 7 wherein said second oligomer comprises a sense oligomer.

9. The composition of claim 7 wherein said second oligomer has a plurality of ribose nucleotide units.

10. The composition of claim 1 wherein said first oligomer includes said nucleotide having said modification.

11. The composition of claim 1 wherein the modification is a morpholino nucleic acid.

12. The composition of claim 11 wherein the oligomer comprises at least one monomer of the formula: 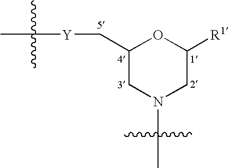
where
Y is a linking group; and
R^{1′} is hydrogen, hydroxy, (C₁-C₄)alkanoyl, naturally occurring nucleobases, non-naturally occurring nucleobases, aromatic moieties, DNA intercalators, nucleobase-binding groups, and heterocyclic moieties, reporter ligands.

13. The composition of claim 12 wherein at least one R^{1′} is a naturally occurring nucleobases or non-naturally occurring nucleobase.

14. The composition of claim 12 wherein Y is a phosphate, phosphorothioate; phosphorodithioate; phosphonate; phosphonothioate; phosphotriester; phosphorothiotriester; phosphoramidate; phosphorothioamidate; phosphinate; and boronate linkage.

15. The composition of claim 12 wherein Y is an ether-, allyl ether-, allyl sulfide-, formacetal/ketal- sulfide-, sulfoxide-, sulfone-, sulfamate-, sulfonamide-, siloxane-, amide-, cationic alkylpolyamine-, guanidyl-, morpholino-, hexose sugar or amide-containing linkage, or a two to four atom linkage containing C, N, O, or S atoms.

16. The composition of claim 12 wherein Y is —NHC(═O)—O—, —CH₂CH₂—O—, —CH₂C(═O)—NH—, —SO₂—N(CH₃)—, N-alkylphoramidite, phosphothioate, or phosphate.

17. The composition of claim 1 wherein the modification is a peptide nucleic acid.

18. The composition of claim 17 wherein the peptide nucleic acid is of the formula: 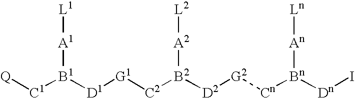
wherein:
n is at least 2,
each of L¹-Lⁿ is independently selected from the group consisting of hydrogen, hydroxy, (C₁-C₄)alkanoyl, naturally occurring nucleobases, non-naturally occurring nucleobases, aromatic moieties, DNA intercalators, nucleobase-binding groups, heterocyclic moieties, and reporter ligands, at least one of L¹-Lⁿ being a naturally occurring nucleobase, a non-naturally occurring nucleobase, a DNA intercalator, or a nucleobase-binding group;
each of A¹-Aⁿ is a single bond, a methylene group or a group of formula: 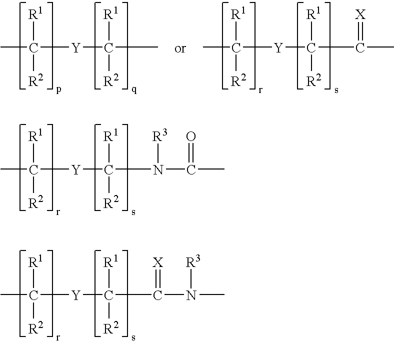
where:
X is O, S, Se, NR , CH₂ or C(CH₃)₂;
Y is a single bond, O, S or NR⁴;
each of p and q is zero or an integer from 1 to 5, the sum p+q being not more than 10;
each of r and s is zero or an integer from 1 to 5, the sum r+s being not more than 10;
each R¹ and R² is independently selected from the group consisting of hydrogen, (C₁-C₄)alkyl which may be hydroxy- or alkoxy- or alkylthio-substituted, hydroxy, alkoxy, alkylthio, amino and halogen; and
each R³ and R⁴is independently selected from the group consisting of hydrogen, (C₁-C₄)alkyl, hydroxy- or alkoxy- or alkylthio-substituted (C₁-C₄)alkyl, hydroxy, alkoxy, alkylthio and amino;
each of B¹-Bⁿ is N or R³N⁺, where R³is as defined above;
each of C¹-Cⁿ is CR⁶R⁷, CHR⁶CHR⁷ or CR⁶R⁷CH₂, where R⁶ is hydrogen and R⁷ is selected from the group consisting of the side chains of naturally occurring alpha amino acids, or R⁶ and R⁷ are independently selected from the group consisting of hydrogen, (C₂-C₆)alkyl, aryl, aralkyl, heteroaryl, hydroxy, (C₁-C₆)alkoxy, (C₁-C₆)alkylthio, NR³R⁴ and SR⁵, where R³ and R⁴ are as defined above, and R⁵ is hydrogen, (C₁-C₆)alkyl, hydroxy-, alkoxy-, or alkylthio-substituted (C₁-C₆)alkyl, or R⁶ and R⁷ taken together complete an alicyclic or heterocyclic system;
each of D¹-Dⁿ is CR⁶R⁷, CH₂CR⁶R⁷ or CHR⁶CHR⁷, where R⁶ and R⁷ are as defined above;
each of G¹-Gⁿ⁻¹ is —NR³CO—, —NR³CS—, —NR³SO— or —NR³SO²⁻, in either orientation, where R³is as defined above;
Q is —CO₂H, —CONR′ R″, —SO₃H or —SO₂NR′R″ or an activated derivative of —CO₂H or —SO₃H; and
I is —NHR′″R″″or —NR′″C(O)R″″, where R′, R″, R′″ and R″″ are independently selected from the group consisting of hydrogen, alkyl, amino protecting groups, reporter ligands, intercalators, chelators, peptides, proteins, carbohydrates, lipids, steroids, oligomers and soluble and non-soluble polymers.

19. The composition of claim 17 wherein the peptide nucleic acid is of the formula: 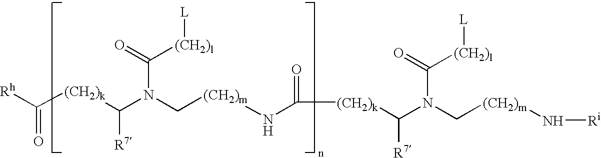
wherein:
each L is independently selected from the group consisting of hydrogen, phenyl, heterocyclic moieties, naturally occurring nucleobases, and non-naturally occurring nucleobases;
each R^{7′} is independently selected from the group consisting of hydrogen and the side chains of naturally occurring alpha amino acids;
n is an integer from 1 to 60,
each k and m is, independently, zero or 1;
each l is zero or an integer from 1 to 5;
R^{h} is OH, NH₂ or —NHLysNH₂; and
Rⁱ is H or COCH₃.

20. The composition of claim 17 wherein the peptide nucleic acid is of the formula: 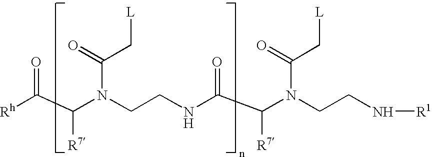
each L is independently selected from the group consisting of the nucleobases
each R^{7′} is hydrogen; and
n is an integer from 1 to 30.

21. The composition of claim 20 wherein n is from 20 to about 23.

22. The composition of claim 17 wherein the peptide nucleic acid is of the formula: 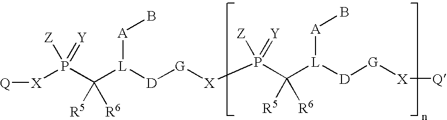
wherein:
n is a number from zero to 100;
A independently of one another is a single bond, a methylene group or a group of formula 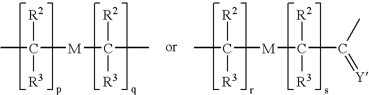
in which M is a single bond, —O—, —S—or —NR¹—, where R¹ is hydrogen or (C₁-C₆)-alkyl optionally substituted by hydroxyl, (C₁-C₆)-alkoxy, (C₁-C₆)-alkylthio or amino;
R² and R³ independently of one another are hydrogen, hydroxyl, (C₁-C₆)-alkoxy, (C₁-C₆)-alkylthio, amino, halogen, or (C₁-C₆)-alkyl optionally substituted by hydroxyl, (C₁-C₆)-alkoxy or (C₁-C₆)-alkylthio;
p and q independently of one another are zero to 5; and
r and s independently of one another are zero to 5;
B independently of one another is hydrogen, hydroxyl, (C₁-C₂₀)-alkyl, (C₁-C₂₀)-alkoxy, (C₁-C₂₀)-alkylthio, (C₆-C₂₀)-aryl-(C₁-C₆)-alkyl, (C₆-C₂₀)-aryl-(C₁-C₆)-alkoxy, (C₆-C₂₀)-aryl-(C₁-C₆)-alkythio, an aromatic group or a heterocyclic group, wherein the alkyl, alkyl portion of alkoxy or alkylthio, aromatic or heterocyclic group is optionally substituted one or more times by hydroxyl, (C₁-C₄)-alkoxy, —NR⁹R¹⁰, oxo, —C(O)OR⁸, —C(O)NR⁹R¹⁰, —CN, —F, —Cl, —Br, —NO₂, (C₂-C₆)-alkoxyalkyl, —S(O)ₘR⁸, —(C₁-C₆)—alkyl—S(O)ₘR⁸, —NHC(═NH)NHR⁸, —C(═NH)NHR⁹, NR⁹C(═O)R⁸, =NOR⁸, NR⁹C(═O)OR¹⁰, —OC(═O)NR⁹R¹⁰, —NR⁹C(═O)NR⁹R¹⁰, a natural nucleobase, an unnatural nucleobase or a reporter ligand, with the proviso that at least one B moiety is a nucleobase;
m is zero, 1 or 2; or,
A-B independent of other A and B groups, can be a D- or L-amino acid condensed on via the carboxyl group or a peptide containing amino acids having a length of up to 5 amino acid residues, with the proviso that at least one B moiety is a nucleobase;
L independently of one another is N or R¹N⁺, where R¹ is as defined above; and
Y′ is ═O, ═S, ═CH₂, ═C(CH₃)₂ or ═NR¹, where R¹ is as defined above;
D and G each independently represent CR⁵R⁶ which can be the same or different;
R⁵ and R⁶ independently of one another are hydrogen, (C₁-C₆)-alkyl, (C₆-C₂₀)-aryl, (C₆-C₂₀)-aryl-(C₁-C₆)-alkyl, hydroxyl, (C₁-C₆)-alkoxy, (C₁-C₆)-alkylthio, wherein the alkyl, alkyl portion of alkoxy or alkylthio, or aryl group is optionally substituted by SR¹ or NR¹R¹, where R¹ is as defined above and R¹ independently of R¹ has the same meaning as R¹;
X independently of one another is —O—, —S—or —NR¹—, in which R¹ is as defined above;
Y independently of one another is ═O or ═S;
Z independently of one another is —OR⁸, —NR⁹R¹⁰ or X′Q″, where X′ is defined as X above and Q″ is defined as Q below;
R⁸ is hydrogen, (C₁-C₁₈)-alkyl, (C₂-C₁₈)-alkenyl, (C₃-C₁₈)-alkynyl, (C₆-C₁₂)-aryl, (C₆-C₁₂)-aryl-(C₁-C₆)-alkyl, wherein alkyl is optionally substituted one or more times by hydroxyl, (C₁-C₄)-alkoxy, F, Cl or Br and wherein aryl is optionally substituted 1-3 times by hydroxyl, (C₁-C₄)-alkoxy, (C₁-C₄)-alkyl, F, Cl, Br, NO₂, —NR⁹R¹⁰, —C(O)OH, —C(O)O—(C₁-C₆)-alkyl or —C(O)NR⁹R¹⁰;
R⁹ and R¹⁰ independently of one another are hydrogen, (C₁-C₁₈)-alkyl, (C₂-C₁₈)-alkenyl, (C₃-C₁₈)-alkynyl, (C₆-C₁₂)-aryl, (C₆-C₁₂)-aryl-(C₁-C₆)-alkyl, where alkyl is optionally substituted one or more times by hydroxyl, (C₁-C₄)-alkoxy, F, Cl or Br; or R⁹ and R¹⁰ form a 4 to 7-membered ring together with the N atom in —NR⁹R¹⁰;
Q and Q′ independently of one another are R⁸, modified or unmodified oligonucleotides or conjugates which a) favorably affect the properties of antisense oligonucleotides, b) affect the properties of triple helix-forming oligonucleotides, c) serve as a label of a DNA probe, or d) during the hybridization of the oligonucleotide analog to the target nucleic acid, attack the target nucleic acid with binding or cross-linking; or Q and Q′ alone or together are a single bond in a cyclic molecule; or Q and Q′, when neither is hydrogen, can be linked together to form a cyclic molecule.

23. The composition of claim 17 wherein the peptide nucleic acid is of the formula: 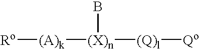
in which B—X is 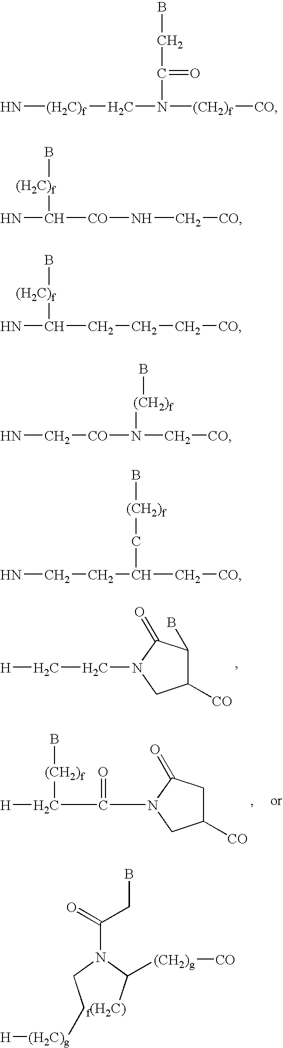
where f is 1-4 and g is 0-3; R^{o} is hydrogen, C₁-C₁₈-alkanoyl, C₁-C₁₈-alkoxy-C₃-C₈-cycloalkanoyl, C₇-C₁₅-aroyl, C₃-C₁₃-heteroaroyl, or a group which favors intracellular uptake of the oligomer;
A is an amino acid radical;
k is an integer from zero to 10;
Q is an amino acid radical;
lis an integer from zero to 10;
B is a natural nucleotide base or unnatural nucleotide base conventionally used in nucleotide chemistry or their prodrug forms, or a base substitute compound;
Q^{o} is hydroxyl, NH₂ or NHR″, in which R″ is C₁-C₁₈-alkyl, C₂-C₁₈-aminoalkyl or C₂-C₁₈-hydroxyalkyl; and
n is an integer from 1-50.

24. The composition of claim 17 wherein the peptide nucleic acid is of the formula: 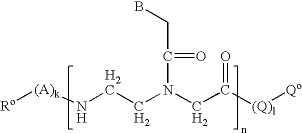
wherein:
R^{o} is hydrogen, C₁-C₁₈-alkanoyl, C₁-C₁₈-alkoxycarbonyl, C₃-C₈-cycloalkanoyl, C₇-C₁₅-aroyl, C₃-C₁₃-heteroaroyl, or a group which favors intracellular uptake of the oligomer;
A is an amino acid residue;
k is an integer from zero to 10;
Q is an amino acid residue;
m is an integer from 0 to 20;
B is a nucleotide base;
Q^{o} is hydroxyl, NH₂ or NHR″, with R″ is C₁-C₁₈-alkyl, C₂-C₁₈-aminoalkyl or C₂-C₁₈-hydroxyalkyl; and
n is an integer of 1-50.

25. The composition of claim 17 wherein the peptide nucleic acid is of the formula:
-LQM-
wherein
Q is a linker or chemical bond;
one of L and M is a nucleotide moiety of the formula: 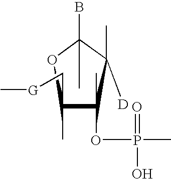
where B is a natural or unnatural nucleobase comprising a bond linking a nucleobase protecting group to the natural or unnatural nucleobase; and
D is a hydrogen atom, a hydroxyl group, a methoxyl group or a hydroxyl group which is protected by a protecting group;
the other of L and M is a PNA moiety of the formula: 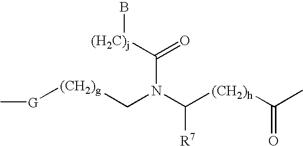
wherein G is a secondary nitrogen atom, a tertiary nitrogen atom having an alkyl substituent, an oxygen atom or a sulfur atom;
B is a natural or unnatural nucleobase;
R⁷ is selected from the group consisting of hydrogen and a side chain of a protected or unprotected naturally occurring α-amino acid; and
each of j, g and h is the same or different and is independently zero or an integer from one to five.

26. The composition of claim 17 wherein the peptide nucleic acid is of the formula: 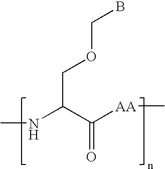
where B is a nucleobase, AA is an amino acid, and n is an integer greater than 1.

27. The composition of claim 1 wherein the modification is of the formula:
X—(B-L)ₙ-B—Y
where B is a nucleobase capable of effecting Watson/Crick base-pairing and bearing two linking attachement sites;
L is a linker comprising 4-7 bonds;
X and Y are terminating groups; and n is an integer equal to 1 or greater.

28. The composition of claim 1 wherein the modification is of formula: 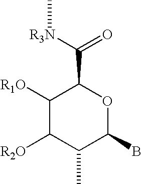
wherein R₁ and R₂ are, independently, hydrogen, lower alkyl or acyl and R₃ is hydrogen or lower alkyl.

29. The composition of claim 1 wherein the modification is of the formula: 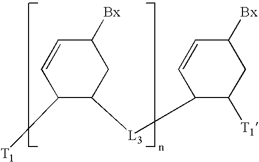
wherein
each Bx is a heterocyclic base moiety;
T₁ is hydroxyl or a protected hydroxyl;
T_{1′} is hydroxyl or a protected hydroxyl; and
L₃ is an internucleoside linkage.

31. The composition of claim 1 wherein the modification is of the formula: 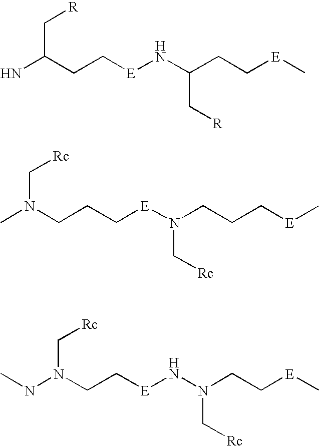
where E is C(═O) or SO₂; and
R and Rc are each independently selected from the group consisting of hydrogen, hydroxy, (C₁-C₄)alkanoyl, naturally occurring nucleobases, non-naturally occurring nucleobases, aromatic moieties, DNA intercalators, nucleobase-binding groups, heterocyclic moieties, and reporter ligands, at least one of R and Rc being a naturally occurring nucleobase, a non-naturally occurring nucleobase, a DNA intercalator, or a nucleobase-binding group.

30. The composition of claim 29 wherein at least one R or Rc is a naturally occurring nucleobase, a non-naturally occurring nucleobase.

32. A composition comprising an oligomer complementary to and capable of hybridizing to a selected target nucleic acid and at least one protein, said protein comprising at least a portion of a RNA-induced silencing complex (RISC), wherein:
said oligomer includes at least one nucleotide having a modification comprising a peptide nucleic acid, a peptide nucleic acid mimic, a morpholino nucleic acid, hexose sugar with amide linkage, cyclohexenyl nucleic acid (CeNA)or an acyclic backbone moiety.

33. The composition of claim 32 wherein said oligomer is an antisense oligomer.

34. The composition of claim 32 wherein said oligomer has 10 to 40 nucleotides.

35. The composition of claim 32 wherein said oligomer has 18 to 30 nucleotides.

36. The composition of claim 32 wherein said oligomer has 21 to 24 nucleotides.

37. The composition of claim 32 further including a further oligomer, wherein said further oligomer is complementary to said oligomer.

38. The composition of claim 37 wherein said further oligomer is a sense oligomer.

39. The composition of claim 37 wherein said further oligomer is an oligomer having a plurality of ribose nucleotide units.

40. The composition of claim 32 wherein the modification is a morpholino nucleic acid.

41. The composition of claim 40 wherein the oligomer comprises at least one monomer of the formula: 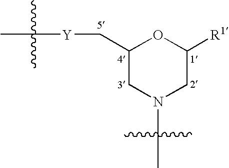
where
Y is a linking group; and
R^{1′} is hydrogen, hydroxy, (C₁-C₄)alkanoyl, naturally occurring nucleobases, non-naturally occurring nucleobases, aromatic moieties, DNA intercalators, nucleobase-binding groups, and heterocyclic moieties, reporter ligands.

42. The composition of claim 41 wherein at least one R^{1′} is a naturally occurring nucleobases or non-naturally occurring nucleobase.

43. The composition of claim 41 wherein Y is a phosphate, phosphorothioate; phosphorodithioate; phosphonate; phosphonothioate; phosphotriester; phosphorothiotriester; phosphoramidate; phosphorothioamidate; phosphinate; and boronate linkage.

44. The composition of claim 41 wherein Y is an ether-, allyl ether-, allyl sulfide-, formacetal/ketal- sulfide-, sulfoxide-, sulfone-, sulfamate-, sulfonamide-, siloxane-, amide-, cationic alkylpolyamine-, guanidyl-, morpholino-, hexose sugar or amide-containing linkage, or a two to four atom linkage containing C, N, O, or S atoms.

45. The composition of claim 41 wherein Y is —NHC(═O)—O—, —CH₂CH₂—O—, —CH₂C(═O)—NH—, —SO₂—N(CH₃)—, N-alkylphoramidite, phosphothioate, or phosphate.

46. The composition of claim 32 wherein the modification is a peptide nucleic acid.

47. The composition of claim 46 the peptide nucleic acid is of the formula: 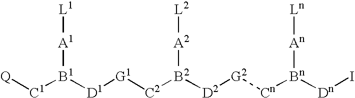
wherein:
n is at least 2,
each of L¹-Lₙ is independently selected from the group consisting of hydrogen, hydroxy, (C₁-C₄)alkanoyl, naturally occurring nucleobases, non-naturally occurring nucleobases, aromatic moieties, DNA intercalators, nucleobase-binding groups, heterocyclic moieties, and reporter ligands, at least one of L¹-Lⁿ being a naturally occurring nucleobase, a non-naturally occurring nucleobase, a DNA intercalator, or a nucleobase-binding group;
each of A¹-Aⁿ is a single bond, a methylene group or a group of formula: 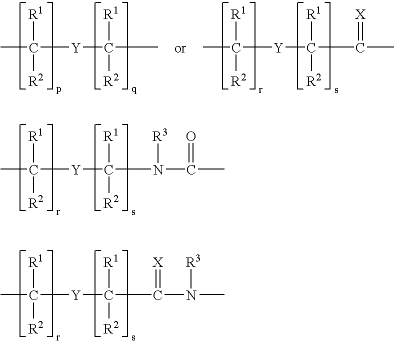
where:
X is O, S, Se, NR³, CH₂ or C(CH₃)₂;
Y is a single bond, O, S or NR⁴;
each of p and q is zero or an integer from 1 to 5, the sum p+q being not more than 10;
each of r and s is zero or an integer from 1 to 5, the sum r+s being not more than 10;
each R¹ and R² is independently selected from the group consisting of hydrogen, (C₁-C₄)alkyl which may be hydroxy- or alkoxy- or alkylthio-substituted, hydroxy, alkoxy, alkylthio, amino and halogen; and
each R³ and R⁴ is independently selected from the group consisting of hydrogen, (C₁-C₄)alkyl, hydroxy- or alkoxy- or alkylthio-substituted (C₁-C₄)alkyl, hydroxy, alkoxy, alkylthio and amino;
each of B¹-Bⁿ is N or R³N⁺, where R³ is as defined above;
each of C¹-Cⁿ is CR⁶R⁷, CHR⁶CHR⁷ or CR⁶R⁷CH₂, where R⁶ is hydrogen and R⁷ is selected from the group consisting of the side chains of naturally occurring alpha amino acids, or R⁶ and R⁷ are independently selected from the group consisting of hydrogen, (C₂-C₆)alkyl, aryl, aralkyl, heteroaryl, hydroxy, (C₁-C₆)alkoxy, (C₁-C₆)alkylthio, NR³R⁴ and SR⁵, where R³ and R⁴ are as defined above, and R⁵ is hydrogen, (C₁-C₆)alkyl, hydroxy-, alkoxy-, or alkylthio-substituted (C₁-C₆)alkyl, or R⁶ and R⁷ taken together complete an alicyclic or heterocyclic system;
each of D¹-Dⁿ is CR⁶R⁷, CH₂CR⁶R⁷ or CHR⁶CHR⁷, where R⁶ and R⁷ are as defined above;
each of G¹-Gⁿ⁻¹ is —NR³CO—, —NR³CS—, —NR³SO— or —NR³SO²⁻, in either orientation, where R³ is as defined above;
Q is —CO₂H, —CONR′R″, —SO₃H or —SO₂NR′R″ or an activated derivative of —CO₂H or —SO₃H; and
I is —NHR′″R″″ or —NR′″C(O)R″″, where R′, R″, R′″ and R″″ are independently selected from the group consisting of hydrogen, alkyl, amino protecting groups, reporter ligands, intercalators, chelators, peptides, proteins, carbohydrates, lipids, steroids, oligonucleotides and soluble and non-soluble polymers.

48. The composition of claim 46 wherein the peptide nucleic acid is of the formula: 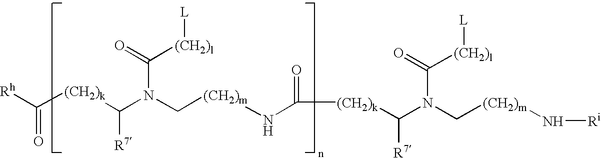
wherein:
each L is independently selected from the group consisting of hydrogen, phenyl, heterocyclic moieties, naturally occurring nucleobases, and non-naturally occurring nucleobases;
each R^{7′} is independently selected from the group consisting of hydrogen and the side chains of naturally occurring alpha amino acids;
n is an integer from 1 to 60,
each k and m is, independently, zero or 1;
each l is zero or an integer from 1 to 5;
R^{h} is OH, NH₂ or —NHLysNH₂; and
Rⁱ is H or COCH₃.

49. The composition of claim 46 the peptide nucleic acid is of the formula: 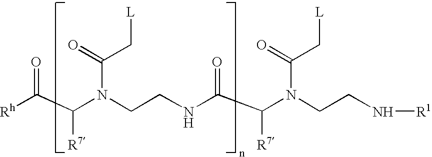
each L is independently selected from the group consisting of the nucleobases
each R^{7′} is hydrogen; and
n is an integer from 1 to 30.

50. The composition of claim 49 wherein n is from 20 to about 23.

51. The composition of claim 46 wherein the peptide nucleic acid is of the formula: 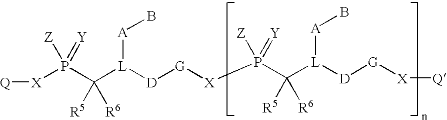
wherein:
n is a number from zero to 100;
A independently of one another is a single bond, a methylene group or a group of formula 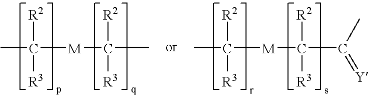
in which M is a single bond, —O—, —S—or —NR¹—, where R¹ is hydrogen or (C₁-C₆)-alkyl optionally substituted by hydroxyl, (C₁-C₆)-alkoxy, (C₁-C₆)-alkylthio or amino;
R² and R³ independently of one another are hydrogen, hydroxyl, (C₁-C₆)-alkoxy, (C₁-C₆)-alkylthio, amino, halogen, or (C₁-C₆)-alkyl optionally substituted by hydroxyl, (C₁-C₆)-alkoxy or (C₁-C₆)-alkylthio;
p and q independently of one another are zero to 5; and
r and s independently of one another are zero to 5;
B independently of one another is hydrogen, hydroxyl, (C₁-C₂₀)-alkyl, (C₁-C₂₀)-alkoxy, (C₁-C₂₀)-alkylthio, (C₆-C₂₀)-aryl-(C₁-C₆)-alkyl, (C₆-C₂₀)-aryl-(C₁-C₆)-alkoxy, (C₆-C₂₀)-aryl-(C₁-C₆)-alkythio, an aromatic group or a heterocyclic group, wherein the alkyl, alkyl portion of alkoxy or alkylthio, aromatic or heterocyclic group is optionally substituted one or more times by hydroxyl, (C₁-C₄)-alkoxy, —NR⁹R¹⁰, oxo, —C(O)OR⁸, —C(O)NR⁹R¹⁰, —CN, —F, —Cl, —Br, —NO₂, (C₂-C₆)-alkoxyalkyl, —S(O)ₘR⁸, —(C₁-C₆)-alkyl-S(O)ₘR⁸, —NHC(═NH)NHR⁸, —C(═NH)NHR⁹, NR⁹C(═O)R⁸, ═NOR⁸, NR⁹C(═O)OR¹⁰, —OC(═O)NR⁹R¹⁰, —NR⁹C(═O)NR⁹R¹⁰, a natural nucleobase, an unnatural nucleobase or a reporter ligand, with the proviso that at least one B moiety is a nucleobase;
m is zero, 1 or 2; or,
A-B independent of other A and B groups, can be a D- or L-amino acid condensed on via the carboxyl group or a peptide containing amino acids having a length of up to 5 amino acid residues, with the proviso that at least one B moiety is a nucleobase;
L independently of one another is N or R¹N⁺, where R¹ is as defined above; and
Y′ is ═O, ═S, ═CH₂, ═C(CH₃)₂ or ═NR¹, where R¹ is as defined above;
D and G each independently represent CR⁵R⁶ which can be the same or different;
R⁵ and R⁶ independently of one another are hydrogen, (C₁-C₆)-alkyl, (C₆-C₂₀)-aryl, (C₆-C₂₀)-aryl-(C₁-C₆)-alkyl, hydroxyl, (C₁-C₆)-alkoxy, (C₁-C₆)-alkylthio, wherein the alkyl, alkyl portion of alkoxy or alkylthio, or aryl group is optionally substituted by SR¹ or NR¹R¹, where R¹ is as defined above and R¹ independently of R¹ has the same meaning as R¹;
X independently of one another is —O—, —S—or —NR¹—, in which R¹ is as defined above;
Y independently of one another is ═O or ═S;
Z independently of one another is —OR⁸, —NR⁹R¹⁰ or X′Q″, where X′ is defined as X above and Q″ is defined as Q below;
R⁸ is hydrogen, (C₁-C₁₈)-alkyl, (C₂-C₁₈)-alkenyl, (C₃-C₁₈)-alkynyl, (C₆-C₁₂)-aryl, (C₆-C₁₂)-aryl-(C₁-C₆)-alkyl, wherein alkyl is optionally substituted one or more times by hydroxyl, (C₁-C₄)-alkoxy, F, Cl or Br and wherein aryl is optionally substituted 1-3 times by hydroxyl, (C₁-C₄)-alkoxy, (C₁-C₄)-alkyl, F, Cl, Br, NO₂, —NR⁹R¹⁰, —C(O)OH, —C(O)O—(C₁-C₆)-alkyl or —C(O)NR⁹R¹⁰;
R⁹ and R¹⁰ independently of one another are hydrogen, (C₁-C₁₈)-alkyl, (C₂-C₁₈)-alkenyl, (C₃-C₁₈)-alkynyl, (C₆-C₁₂)-aryl, (C₆-C₁₂)-aryl-(C₁-C₆)-alkyl, where alkyl is optionally substituted one or more times by hydroxyl, (C₁-C₄)-alkoxy, F, Cl or Br; or R⁹ and R¹⁰ form a 4 to 7-membered ring together with the N atom in —NR⁹R¹⁰;
Q and Q′ independently of one another are R⁸, modified or unmodified oligonucleotides or conjugates which a) favorably affect the properties of antisense oligonucleotides, b) affect the properties of triple helix-forming oligonucleotides, c) serve as a label of a DNA probe, or d) during the hybridization of the oligonucleotide analog to the target nucleic acid, attack the target nucleic acid with binding or cross-linking; or Q and Q′ alone or together are a single bond in a cyclic molecule; or Q and Q′, when neither is hydrogen, can be linked together to form a cyclic molecule.

52. The composition of claim 46 wherein the peptide nucleic acid is of the formula: 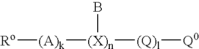
in which B—X is 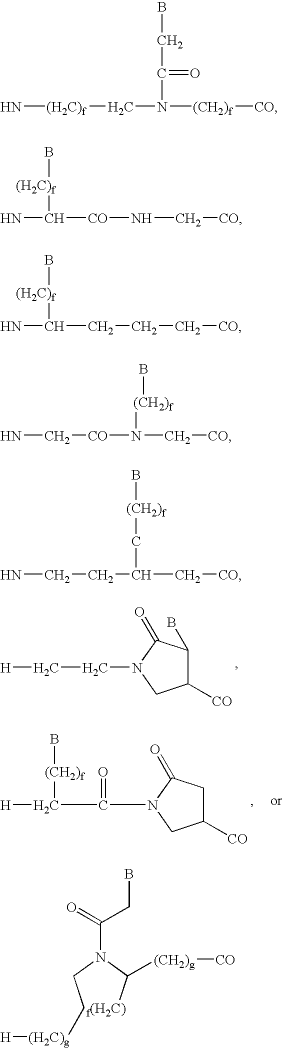
where f is 1-4 and g is 0-3; R^{o} is hydrogen, C₁-C₁₈-alkanoyl, C₁-C₁₈-alkoxy-carbonyl, C₃-C₈-cycloalkanoyl, C₇-C₁₅-aroyl, C₃-C₁₃-heteroaroyl, or a group which favors intracellular uptake of the oligomer;
A is an amino acid radical;
k is an integer from zero to 10;
Q is an amino acid radical;
l is an integer from zero to 10;
B is a natural nucleotide base or unnatural nucleotide base conventionally used in nucleotide chemistry or their prodrug forms, or a base substitute compound;
Q^{o} is hydroxyl, NH₂ or NHR″, in which R″ is C₁-C₁₈-alkyl, C₂-C₁₈-aminoalkyl or C₂-C₁₈-hydroxyalkyl; and
n is an integer from 1-50.

53. The composition of claim 46 wherein the peptide nucleic acid is of the formula: 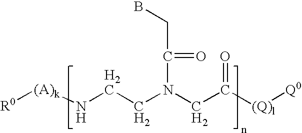
where R^{o} is hydrogen, C₁-C₁₈-alkanoyl, C₁-C₁₈-alkoxycarbonyl, C₃-C₈-cycloalkanoyl, C₇-C₁₅-aroyl, C₃-C₁₃-heteroaroyl, or a group which favors intracellular uptake of the oligomer;
A is an amino acid residue;
k is an integer from zero to 10;
Q is an amino acid residue;
m is an integer from 0 to 20;
B is a nucleotide base;
Q^{o} is hydroxyl, NH₂ or NHR″, with R″ is C₁-C₁₈-alkyl, C₂-C₁₈-aminoalkyl or C₂-C₁₈-hydroxyalkyl; and
n is an integer of 1-50.

54. The composition of claim 46 wherein the peptide nucleic acid is of the formula:
-LQM-
wherein
Q is a linker or chemical bond;
one of L and M is a nucleotide moiety of the formula: 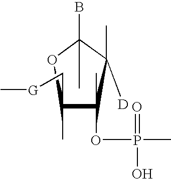
where B is a natural or unnatural nucleobase comprising a bond linking a nucleobase protecting group to the natural or unnatural nucleobase; and
D is a hydrogen atom, a hydroxyl group, a methoxyl group or a hydroxyl group which is protected by a protecting group;
the other of L and M is a PNA moiety of the formula: 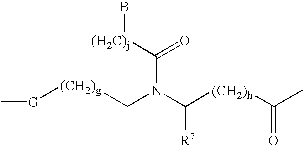
wherein G is a secondary nitrogen atom, a tertiary nitrogen atom having an alkyl substituent, an oxygen atom or a sulfur atom;
B is a natural or unnatural nucleobase;
R⁷ is selected from the group consisting of hydrogen and a side chain of a protected or unprotected naturally occurring α-amino acid; and
each of j, g and h is the same or different and is independently zero or an integer from one to five.

55. The composition of claim 46 wherein the peptide nucleic acid is of the formula: 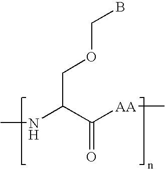
where B is a nucleobase, AA is an amino acid, and n is an integer greater than 1.

56. The composition of claim 32 wherein the modification comprises the formula:
X—(B-L)ₙ-B—Y
where B is a nucleobase capable of effecting Watson/Crick base-pairing and bearing two linking attachement sites;
L is a linker comprising 4-7 bonds;
X and Y are terminating groups; and
n is an integer equal to 1 or greater.

57. The composition of claim 32 wherein the modification is of formula: 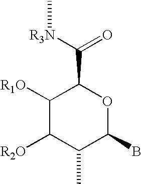
wherein R₁ and R₂ are, independently, hydrogen, lower alkyl or acyl and R₃ is hydrogen or lower alkyl.

58. The composition of claim 32 wherein the modification is of the formula: 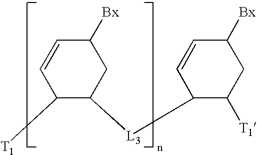
wherein
each Bx is a heterocyclic base moiety;
T₁ is hydroxyl or a protected hydroxyl;
T_{1′} is hydroxyl or a protected hydroxyl; and
L₃ is an internucleoside linkage.

59. The composition of claim 32 wherein the modification comprises the formula: 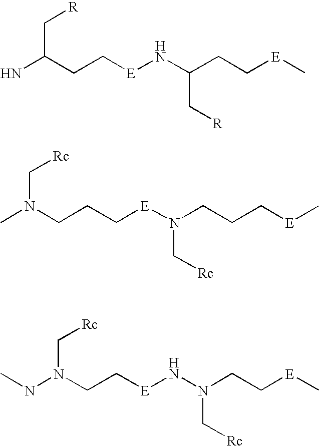
where E is C(═O) or SO₂; and
R and Rc are each independently selected from the group consisting of hydrogen, hydroxy, (C₁-C₄)alkanoyl, naturally occurring nucleobases, non-naturally occurring nucleobases, aromatic moieties, DNA intercalators, nucleobase-binding groups, heterocyclic moieties, and reporter ligands, at least one of R and Rc being a naturally occurring nucleobase, a non-naturally occurring nucleobase, a DNA intercalator, or a nucleobase-binding group.

60. The composition of claim 59 wherein at least one R or Rc is a naturally occurring nucleobase, a non-naturally occurring nucleobase.

61. An oligomer having at least a first region and a second region, wherein:
said first region of said oligomer is complementary to and capable of hybridizing with said second region of said oligomer,
at least a portion of said oligomer is complementary to and capable of hybridizing to a selected target nucleic acid, and
said oligomer further includes at least one nucleotide having a modification comprising a peptide nucleic acid, a peptide nucleic acid mimic, a morpholino nucleic acid or an acyclic backbone moiety.

62. The oligomer of claim 61 wherein each of said first and said second regions has at least 10 nucleotides.

63. The oligomer of claim 61 wherein said first regions in a 5′ to 3′ direction is complementary to said second region in a 3′ to 5′ direction.

64. The oligomer of claim 61 wherein said oligomer includes a hairpin structure.

65. The oligomer of claim 61 wherein said first region of said oligomer is spaced from said second region of said oligomer by a third region and where said third region comprises at least two nucleotides.

66. The oligomer of claim 61 wherein said first region of said oligomer is spaced from said second region of said oligomer by a third region and where said third region comprises a non-nucleotide region.

67. A pharmaceutical composition comprising the composition of claim 1 and a pharmaceutically acceptable carrier.

68. A pharmaceutical composition comprising the composition of claim 32 and a pharmaceutically acceptable carrier.

69. A pharmaceutical composition comprising the oligomeric compound of claim 61 and a pharmaceutically acceptable carrier.

70. A method of modulating the expression of a target nucleic acid in a cell comprising contacting said cell with a composition of claim 1.

71. A method of modulating the expression of a target nucleic acid in a cell comprising contacting said cell with a composition of claim 32.

72. A method of modulating the expression of a target nucleic acid in a cell comprising contacting said cell with an oligomeric compound of claim 61.

73. A method of treating or preventing a disease or disorder associated with a target nucleic acid comprising administering to an animal having or predisposed to said disease or disorder a therapeutically effective amount of a composition of claim 1.

74. A method of treating or preventing a disease or disorder associated with a target nucleic acid comprising administering to an animal having or predisposed to said disease or disorder a therapeutically effective amount of a composition of claim 32.

75. A method of treating or preventing a disease or disorder associated with a target nucleic acid comprising administering to an animal having or predisposed to said disease or disorder a therapeutically effective amount of a composition of claim 61.


SEQUENCE LISTING (6 sequences)

SEQ ID NO: 1 - DNA, 19 bp, Artificial Sequence
  Oligonucleotide
cgagaggcggacgggaccg

SEQ ID NO: 2 - DNA, 21 bp, Artificial Sequence
  Oligonucleotide
cgagaggcggacgggaccgtt

SEQ ID NO: 3 - DNA, 21 bp, Artificial Sequence
  Oligonucleotide
cggtcccgtccgcctctcgtt

SEQ ID NO: 4 - DNA, 20 bp, Artificial Sequence
  Oligonucleotide
tccgtcatcgctcctcaggg

SEQ ID NO: 5 - DNA, 20 bp, Artificial Sequence
  Oligonucleotide
gtgcgcgcgagcccgaaatc

SEQ ID NO: 6 - DNA, 20 bp, Artificial Sequence
  Oligonucleotide
atgcattctgcccccaagga